(19) United States
(12) Patent Application Publication
(10) Pub. No.: US 2004/0248913 A1
(43) Pub. Date: Dec. 9, 2004

(54) (Pyridinyl and pyrimidyl) trienoic acid derivatives as retinoid x receptor modulators

(76) Inventors: Timothy Alan Grese, Indianapolis, IN (US); Kevin M Gardinier, Indianapolis, IN (US); Marcus F Boehm, San Diego, CA (US)

Correspondence Address: Steven G Davis, Hamilton Brook Smith & Reynolds, P O Box 9133, 530 Virginia Road, Concord, MA, 01742-9133, US

(21) Appl. No.: 10/483,160

(22) PCT Filed: Jul. 18, 2002
(86) PCT No.: PCT/US02/23017

Related U.S. Application Data
(60) Provisional application No. 60/306,951, filed on Jul. 20, 2001.

(51) Int. Cl. A61K 31/513; A61K 31/4439; C 07D 4 3/02; C 07D 4 1/02

(52) U.S. Cl. 514/269; 514/345; 544/314; 546/268.1; 546/288

(57) ABSTRACT

The present invention relates to a method of modulating retinoid X receptor activity in a mammal, novel compounds and pharmaceutical compositions for

modulating retinoid X receptor activity in a mammal, and methods of making compounds that modulate retinoid X receptor activity in a mammal. The compounds are represented by Structural Formula I: The compounds of Structural Formula I are efficacious insulin sensitizers and do not have the undesirable side effects of increasing triglycerides or suppressing the thyroid hormone axis.

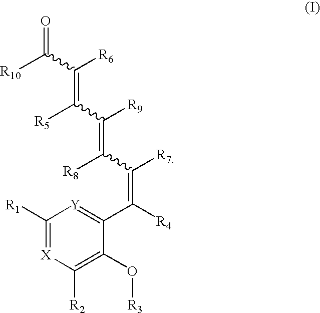

### RELATED APPLICATIONS

This application claims the benefit of U.S. Provisional 60/306,951 filed on 20 Jul. 2001 the entire teachings of which are incorporated herein by reference.

### BACKGROUND OF THE INVENTION

The vitamin A metabolite, retinoic acid, has long been recognized to induce a broad spectrum of biological effects. For example, retinoic acid-containing products, such as Retin-A® and Accutane®, have found utility as therapeutic agents for the treatment of various pathological conditions. In addition, a variety of structural analogues of retinoic acid have been synthesized that also have been found to be bioactive. Many of these synthetic retinoids have been found to mimic many of the pharmacological actions of retinoic acid, and thus have therapeutic potential for the treatment of numerous disease states.

Medical professionals have become very interested in the therapeutic applications of retinoids. Among their uses approved by the FDA is the treatment of severe forms of acne and psoriasis as well as cancers such as Kaposi's Sarcoma. A large body of evidence also exists that these compounds can be used to arrest and, to an extent, reverse the effects of skin damage arising from prolonged exposure to the sun. Other evidence exists that these compounds have clear effects on cellular proliferation, differentiation and programmed cell death (apoptosis), and thus may be useful in the treatment and prevention of a variety of cancerous and pre-cancerous conditions, such as acute promyleocytic leukemia (APL), epithelial cancers, squamous cell carcinomas, including cervical and skin cancers and renal cell carcinoma. Furthermore, retinoids may have beneficial activity in treating and preventing diseases of the eye, cardiovascular disease and other skin disorders.

Major insight into the molecular mechanism of retinoic acid signal transduction was gained in 1988, when a member of the steroid/thyroid hormone intracellular receptor superfamily was shown to transduce a retinoic acid signal. V. Giguere et al., Nature, 330:624-29 (1987); M. Petkovich et al., Nature, 330: 444-50 (1987); for a review, see R. M. Evans, Science, 240:889-95 (1988). It is now known that retinoids regulate the activity of two distinct intracellular receptor subfamilies: the Retinoic Acid Receptors (RARs) and the Retinoid X Receptors (RXFs), including their subtypes, RARα, β, γ and RXRα, β, γ. All-trans-retinoic acid (ATRA) is an endogenous low-molecular-weight ligand that modulates the transcriptional activity of the RARs, while 9-cis retinoic acid (9-cis) is the endogenous ligand for the RXRs. R. A. Heyman et al., Cell, 68:397-406 (1992); and A. A. Levin et al., Nature, 355:359-61 (1992).

Although both the RARs and RXRs respond to ATRA in vivo, due to the in vivo conversion of some of the ATRA to 9-cis, the receptors differ in several important aspects. First, the RARs and RXRs are significantly divergent in primary structure (e.g., the ligand binding domains of RARα and RXRα have only approximately 30% amino acid homology). These structural differences are reflected in the different relative degrees of responsiveness of RARs and RXRs to various vitamin A metabolites and synthetic retinoids. In addition, distinctly different patterns of tissue distribution are seen for RARs and RXRs. For example, RXRα mRNA is expressed at high levels in the visceral tissues, e.g., liver, kidney, lung, muscle and intestine, while RARα mRNA is not. Finally, the RARs and RXRs have different target gene specificity. In this regard, RARs and RXRs regulate transcription by binding to response elements in target genes that generally consist of two direct repeat half-sites of the consensus sequence AGGTCA. RAR:RXR heterodimers activate transcription ligand by binding to direct repeats spaced by five base pairs (a DR5) or by two base pairs (a DR2). However, RXR:RXR homodimers bind to a direct repeat with a spacing of one nucleotide (a DR1). D. J. Mangelsdorf et al., “The Retinoid Receptors” in The Retinoids: Biology, Chemistry and Medicine, M. B. Sporn, A. B. Roberts and D. S. Goodman, Eds., Raven Press, New York, N.Y., 2nd Edition (1994). For example, response elements have been identified in the cellular retinal binding protein type II (CRBPII), which consists of a DR1, and in Apolipoprotein AI genes that confer responsiveness to RXR, but not to RAR. Further, RAR has also been shown to repress RXR-mediated activation through the CRBPII RXR response element (D. J. Manglesdorf et al., Cell, 66:555-61 (1991)). Also, RAR specific target genes have been identified, including target genes specific for RAR, (e.g., PRE), that consist of a DR5. These data indicate that two retinoic acid responsive pathways are not simply redundant, but instead manifest a complex interplay.

RXR agonists in the context of an RXR:RXR homodimer display unique transcriptional activity in contrast to the activity of the same compounds through an RXR heterodimer. Activation of a RXR homodimer is a ligand dependent event, i.e., the RXR agonist must be present to bring about the activation of the RXR homodimer. In contrast, RXR working through a heterodimer (e.g., RXR:RAR, RXR:VDR) is often the silent partner, i.e., no RXR agonist will activate the RXR-containing heterodimer without the corresponding ligand for the heterodimeric partner. However, for other heterodimers, (e.g., PPAR:RXR) a ligand for either or both of the heterodimer partners can activate the heterodimeric complex. Furthermore, in some instances, the presence of both an RXR agonist and the agonist for the other heterodimeric partner (e.g., gemfibrizol for PPARα and TTNPB for RARα) leads to at least an additive, and often a synergistic enhancement of the activation pathway of the other IR of the heterodimer pair (e.g., the PPARα pathway). See e.g., WO 94/15902, published Jul. 21, 1994; R. Mulheijee et al., J. Steroid Biochem. Molec. Biol., 51:157-166 (1994); and L. Jow and R. Mukherjee, J. Biol. Chem., 270:3836-40 (1995).

RXR agonists compounds which have been identified so far have exhibited significant therapeutic utility, but they have also exhibited some undesirable side effects, such as elevation of triglycerides and suppression of the thyroid hormone axis (see, e.g., Sherman, S. I. et al., N. Engl. J. Med. 340(14):1075-1079 (1999).

### SUMMARY OF THE INVENTION

The present invention is directed to compounds represented by Structural Formula I and geometric isomers, pharmaceutically acceptable salts, solvates and hydrates thereof:

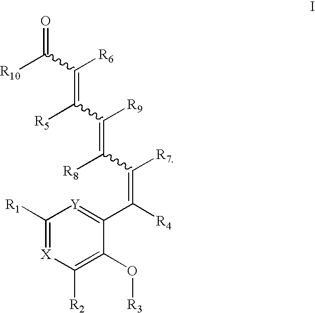

In Structural Formula I, X and Y are each, independently, CH or N, and at least one of X or Y is N. R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂. R₃ is an optionally substituted C₁-C₉ alkyl, a C₁-C₆ haloalkyl, an optionally substituted C₃-C₇ cycloalkyl, or an optionally substituted aralkyl. R₄ and R₅ are each, independently, H, F, an optionally substituted C₁-C₃ alkyl, or a C₁-C₃ haloalkyl. R₆, R₇, R₈, and R₉ are each, independently, H or F. R₁₀ is OR₁₃, OC(O)R₁₄, NR₁₅R₁₆ or an aminoalkoxy. R₁₁, and R₁₂ are each, independently, H or an C₁-C₆ alkyl or taken together with the nitrogen to which they are attached form a heterocycle. R₁₃ is H or a C₁-C₆ alkyl, an aryl or an aralkyl. R₁₄ is a C₁-C₆ alkyl, an aryl or an aralkyl. R₁₅ and R₁₆ are each, independently, H, a C₁-C₆ alkyl, an aryl or an aralkyl.

In one embodiment, the present invention relates to a method of modulating retinoid X receptor activity in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of modulating RXRα:PPARα heterodimer activity in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of modulating RXRα:PPARγ heterodimer activity in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of increasing HDL cholesterol levels and reducing triglyceride levels in a mammal by administering to the mammal a pharmaceutically effective amount of at least one compound represented by Structural Formula I, or a geometric isomer, pharmaceutically acceptable salts, solvates or hydrates thereof.

In another embodiment, the present invention relates to a method of modulating lipid metabolism in a mammal by administering to the mammal a pharmaceutically effective amount of at least one compound represented by Structural Formula L or a geometric isomer, pharmaceutically acceptable salts, solvates or hydrates thereof.

In another embodiment, the present invention relates to a method of lowering blood glucose levels without altering serum triglyceride levels in a mammal by administering to the mammal a pharmaceutically effective amount of at least one compound represented by Structural Formula L or a geometric isomer, pharmaceutically acceptable salts, solvates or hydrates thereof.

In another embodiment, the present invention relates to a method of treating or preventing a disease or condition in a mammal, wherein the disease or condition are selected from the group consisting of syndrome X, non-insulin dependent diabetes mellitus, cancer, photoaging, acne, psoriasis, obesity, cardiovascular disease, atherosclerosis, uterine leiomyomata, inflamatory disease, neurodegenerative diseases, wounds and baldness. The method involves administering to the mammal a pharmaceutically effective amount of at least one compound represented by Structural Formula I, or a geometric isomer, pharmaceutically acceptable salts, solvates or hydrates thereof.

In another embodiment, the present invention also relates to pharmaceutical compositions which include a pharmaceutically acceptable carrier and at least one compound represented by Structural Formula I, or a geometric isomer, pharmaceutically acceptable salts, solvates or hydrates thereof.

In yet another embodiment, the present invention relates to a method of making a compound represented by Structural Formula I.

The compounds of the present invention and geometric isomers, pharmaceutically acceptable salts, solvates and hydrates thereof are effective in treating diseases or conditions that are mediated by retinoid X receptors or heterodimers of retinoid X receptors. Therefore, the compounds of the invention and pharmaceutically acceptable salts, solvates and hydrates thereof are effective in treating syndrome X, non-insulin dependent diabetes mellitus, cancer, photoaging, acne, psoriasis, obesity, cardiovascular disease, atherosclerosis, uterine leiomyomata, inflamatory disease, neurodegenerative diseases, wounds and baldness. In addition, the compounds of the invention exhibit fewer side effects than compounds currently used to treat these conditions.

### DETAILED DESCRIPTION OF THE INVENTION

The term “alkyl”, alone or in combination, means a straight-chain or branched-chain alkyl radical having from 1 to about 10 carbon atoms. Examples of such radical include methyl, ethyl, n-propyl, isopropyl, n-butyl, isobutyl, sec-butyl, tert-butyl, tert-amyl, pentyl, hexyl, heptyl, octyl and the like. Preferably, an alkyl group has from 1 to 6 carbon atoms.

The term “alkenyl”, alone or in combination, means a straight-chain or branched-chain hydrocarbon radical having one or more carbon-carbon double-bonds and having from 2 to about 10 carbon atoms. Examples of alkenyl radicals include ethenyl, propenyl, 1,4-butadienyl and the like. Preferably, an alkenyl group has from 1 to 6 carbon atoms.

The term “alkynyl”, alone or in combination, means a straight-chain or branched-chain hydrocarbon radical having one or more carbon-carbon triple-bonds and having from 2 to about 10 carbon atoms. Examples of alkynyl radicals include ethynyl, propynyl, butynyl and the like. Preferably, an alkynyl group has from 1 to 6 carbon atoms.

The term “aryl”, alone or in combination, means an optionally substituted six-membered carbocyclic aromatic ring systems (e.g. phenyl), fused polycyclic aromatic ring systems (e.g. naphthyl and anthracenyl) and aromatic ring systems fused to carbocyclic non-aromatic ring systems (e.g., 1,2,3,4-tetrahydronaphthyl). Aryl groups include polyaromatic rings and polycyclic ring systems of from two to four, more preferably two to three, and most preferably two rings.

The term “alkoxy”, alone or in combination, means an alky ether radical wherein the term alkyl is defined as above. Examples of alkoxy radicals include methoxy, ethoxy, n-propoxy, iso-propoxy, n-butoxy, iso-butoxy, sec-butoxy, tert-butoxy and the like.

The term “aryloxy”, alone or in combination, means an aryl ether radical wherein the term aryl is defined as above. Examples of aryloxy radicals include phenoxy, benyloxy and the like.

The term “cycloalkyl”, alone or in combination, means a saturated monocyclic, bicyclic or tricyclic alkyl radical wherein each cyclic moiety has about 3 to about 8 carbon atoms.

The term “aralkyl”, alone or in combination, means an alkyl radical as defined above in which one hydrogen atom is replaced by an aryl radical as defined above, such as, for example, benzyl, 2-phenylethyl and the like.

The terms “alkyl”, “alkenyl” and “alkynyl” include straight-chain or branched-chain.

The terms “heteroalkyl”, “heteroalkenyl” and “heteroalkynyl” include optionally substituted C₁-C₁₀ alkyl, C₁-C₁₀ alkenyl and C₁-C₁₀ alkynyl structures, as described above, in which one or more skeletal atoms is oxygen, nitrogen, sulfur, or combinations thereof.

The terms “haloalkyl”, “haloalkenyl” and “haloalkynyl” include C₁-C₁₀ alkyl, C₁-C₁₀ alkenyl and C₁-C₁₀ alkynyl structures, as described above, that are substituted with one or more F, Cl, Br or I, or with combinations thereof.

The terms “fluoroalkyl” includes C₁-C₁₀ alkyl structure, as described above, that is substituted with one or more F.

The term “cycloalkyl” includes optionally substituted C₃-C₇ carbocyclic structures.

The term “carbocyclic” means a cycloalkyl, cycloalkenyl or aryl wherein the cyclic moiety is composed of carbon atoms.

The term “heterocycle” includes optionally substituted, saturated, unsaturated, or aromatic three- to eight-membered cyclic structures wherein the cyclic moiety includes one or more oxygen, nitrogen, sulfur, or combinations thereof.

The term “heteroaryl” refers to optionally substituted five- or six-membered heterocyclic aromatic rings containing one or more heteroatoms. The heterocyclic rings may contain one or more heteroatoms selected from the group consisting of oxygen, nitrogen and sulfur. Heterocyclic rings include polycyclic ring systems of from two to four, more preferably two to three, and most preferably two aromatic rings including, without limitation, furyl, pyrrolyl, pyrrolidinyl, thienyl, pyridyl, piperidyl, indolyl, quinolyl, thiazole, benzthiazole and triazole.

The term “azaaryl” refers to pyridyl and pyrimidyl.

The substituents of an “optionally substituted” structure may include, but are not limited to, one or more of the following preferred substituents: F, Cl, Br, L CN, NO₂, NH₂, NHCH₃, N(CH₃)₂, SH, SCH₃, OH, OCH₃, OCF₃, CH₃, CF₃.

The term “halo” includes to F, Cl, Br or I.

An aminoalkyl group is an alkyl group having from one to six carbon atoms which is substituted with at least one amine represented by —NR₂₁R₂₂, in which R₂, and R₂₂ are each, independently, a C₁-C₆ alkyl, an aryl or an aralkyl, or R₂, and R₂₂ taken together with the nitrogen to which they are attached form a five or six membered heterocycloalkyl.

Protecting groups for aromatic hydroxy groups are known to those skilled in the art. For examples of protecting groups for aromatic hydroxy groups see Greene, et al., Protective Groups in Organic Synthesis (1991), John Wiley & Sons, Inc., pages 143-176, the teachings of which are incorporated herein by reference in their entirety. Preferably, an aromatic hydroxy group is protected by converting it to a methoxymethyl ether (see Id., page 149-150) or a methoxyethoxymethyl ether (see Id., page 151).

The term “RXR modulator” refers to a compound that binds to one or more Retinoid X Receptors and modulates (i.e., increases or decreases the transcriptional activity and/or biological properties of the given receptor dimer) the transcriptional activity of an RXR homodimer (i.e., RXR:RXR) and/or RXR in the context of a heterodimer, including but not limited to heterodimer formation with peroxisome proliferator activated receptors (e.g., RXR:PPARα,β,γ1 or γ2), thyroid receptors (e.g., RXR:TRα or β), vitamin D receptors (e.g., RXR:VDR), retinoic acid receptors (e.g., RXR:RARα,β or γ), NGFIB receptors (e.g., RXR:NGFIB), NURR1 receptors (e.g., RXR:NURR1) LXR receptors (e.g., RXR:LXRα,β), DAX receptors (e.g., RXR:DAX), as well as other orphan receptors that form heterodimers with RXR, as either an agonist, partial agonist and/or antagonist. The particular effect of an RXR modulator as an agonist, partial agonist and/or antagonist will depend upon the cellular context as well as the heterodimer partner in which the modulator compounds acts.

In a first embodiment, the compounds represented by Structural Formula L separately or with their respective pharmaceutical compositions, have R₄ and R₇ in a cis configuration.

In a second embodiment, the compounds represented by Structural Formula I, separately or with their respective pharmaceutical compositions, have R₄ and R₇ in a cis configuration, R₈ and R₉ in a trans configuration and R₅ and R₆ in a trans configuration.

In a third embodiment, X is N and Y is CH in the compounds represented by Structural Formula L separately or with their respective pharmaceutical compositions.

In a fourth embodiment, R₃ is an optionally substituted C₁-C₅ alkyl or a C₂-C₅ fluoroalkyl in the compounds represented by Structural Formula I, separately or with their respective pharmaceutical compositions.

In fifth embodiment, X is N, Y is CH, R₃ is an optionally substituted C₁-C₅ alkyl or a C₂-C₅ fluoroalkyl, R₄ and R₇ are in a cis configuration, R₈ and R₉ in a trans configuration and R₅ and R₆ in a trans configuration in the compounds represented by Structural Formula I, separately or with their respective pharmaceutical compositions.

In sixth embodiment, X is N, Y is CH, R₃ is an optionally substituted C₁-C₅ alkyl or a C₂-C₅ fluoroalkyl, R₄ and R₇ are in a cis configuration, R₈ and R₉ in a trans configuration, R₅ and R₆ in a trans configuration, and R₁ and R₂ are the same in the compounds represented by Structural Formula I, separately or with their respective pharmaceutical compositions.

In seventh embodiment, X is N, Y is CH, R₃ is an optionally substituted C₁-C₅ alkyl or a C₂-C₅ fluoroalkyl, R₄ and R₇ are in a cis configuration, R₈ and R₉ in a trans configuration, R₅ and R₆ in a trans configuration, and R₁ and R₂ are the same and are isopropyl in the compounds represented by Structural Formula I, separately or with their respective pharmaceutical compositions.

In embodiments one through seven, R₁₀ is preferably OH.

The fluoroalkyl in embodiments 4, 6 and 7 can have from one to eleven fluoro groups.

Compounds of the present invention include, but are not limited to, the following group of compounds:

7-(3-butoxy-2,6-diisopropyl-pyridin-4-yl)-3-methyl-octa-2(E),4(E),6(Z)-trienoic acid;

7-(2,6-diisopropyl-3-propoxy-pyridinyl)-3-methyl-octa-2(E),4(E),6(Z)-trienoic acid;

7-(2,6-diisopropyl-3-ethoxy-pyridin-4-yl)-3-methyl-octa-2(E),4(E),6(Z)-trienoic acid;

7-[3-(2,2-difluoro-ethoxy)-2,6-diisopropyl-pyridin-4-yl]-3-methyl-octa-2(E),4(E),6(Z)-trienoic acid; and

7-[2,6-diisopropyl-3-(2,2,2-trifluoro-ethoxy)-pyridin-4-yl]-3-methyl-octa-2(E),4(E),6(Z)-trienoic acid, and

pharmaceutically acceptable salts, solvates and hydrates thereof.

The compounds of Formula I represent a select group of compounds among previously disclosed RXR modulators that have insulin sensitizing activity, but do not suppress the thyroid axis and do not elevate triglycerides. These compounds are heterodimer selective modulators of RXR activity. They bind to RXR with high affinity (generally Kᵢ<50 nM) and produce potent synergistic activation of the RXR:PPARγ heterodimer, but preferably do not synergize with RAR agonists at the RXR:RAR heterodimer. This synergistic activation of PPARγ in vitro is contemplated to be a major determinant of the antidiabetic efficacy of the compounds in vivo.

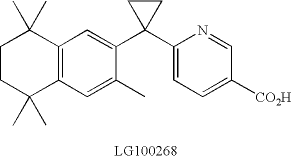

Compounds, such as LG100268, that are full RXR homodimer agonists are efficacious insulin sensitizers in rodent models of Type II Diabetes, but they also raise triglycerides and suppress the thyroid hormone axis.

The compounds of the invention are heterodimer selective modulators of RXR activity. Those compounds that have a carbon chain length at the R₃ position and appropriate substituents at R₁ and R₂ within the scope of the present invention maintain the desirable insulin sensitizing activity and eliminate or reduce both the suppression of the thyroid axis and triglyceride elevations.

The compounds of the invention are expected to be efficacious insulin sensitizers and to eliminate undesirable increases in triglycerides and suppression of T4 because they selectively bind to RXR but do not significantly activate the RXR:RAR heterodimer.

When administered to obese, insulin resistant db/db mice (100 mg/kg by daily oral gavage for 14 days) these heterodimer selective RXR modulators are expected to lower both plasma glucose and triglycerides. However, unlike either full agonists (e.g., LG100268) or partial agonists that exhibit less than 50% activity at the RXR:RAR heterodimer, they are not expected to suppress total circulating levels of T4, or increase triglycerides.

When administered to transgenic mice carrying the human apo A-I gene the compounds of the invention are expected to increase HDL cholesterol, but unlike LG100268 they are not expected to raise triglycerides. These effects are consistent with activation of PPARα, and the compounds of the invention are expected to synergize with PPARα agonists.

The compounds of the present invention possess particular application as RXR modulators and in particular as dimer-selective RXR modulators including, but not limited to, RXR homodimer antagonists, and agonists, partial agonists and antagonists of RXRs in the context of a heterodimer.

In a second aspect, the present invention provides a method of modulating processes mediated by RXR homodimers and/or RXR heterodimers comprising administering to a patient an effective amount of a compound of the invention as set forth above. The compounds of the present invention also include all pharmaceutically acceptable salts, as well as esters and amides. As used in this disclosure, pharmaceutically acceptable salts include, but are not limited to: pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydoxymethyl) aminomethane. Additional pharmaceutically acceptable salts are known to those skilled in the art.

The compounds of the present invention are useful in the modulation of transcriptional activity through RXR in the context of heterodimers other than RXR:RARα,β,γ (e.g., RXR:PPARα,β,γ; RXR:TR; RXR:VDR; RXR:NGFIB; RXR:NURR1; RXR:LXRα,β, RXR:DAX), including any other intracellular receptors (IRs) that form a heterodimer with RXR. For example, application of the compounds of the present invention to modulate a RXRα:PPARα heterodimer is useful to modulate, i.e. increase, HDL cholesterol levels and reduce triglyceride levels. Yet, application of many of the same compounds of the present invention to a RXRα:PPARγ heterodimer modulates a distinct activity, i.e., modulation of adipocyte biology, including effects on the differentiation and apoptosis of adipocytes, which will have implications in the treatment and/or prevention of diabetes and obesity. In addition, use of the modulator compounds of the present invention with activators of the other heterodimer partner (e.g., fibrates for PPARα and thiazolidinediones for PPARγ) can lead to a synergistic enhancement of the desired response. Likewise, application of the modulator compounds of the present invention in the context of a RXRα:VDR heterodimer will be useful to modulate skin related processes (e.g., photoaging, acne, psoriasis), malignant and pre-malignant conditions and programmed cell death (apoptosis). Further, it will be understood by those skilled in the art that the modulator compounds of the present invention will also prove useful in the modulation of other heteromer interactions that include RXR, e.g., trimers, tetramers and the like.

In the context of an RXR homodimer, the compounds of the present invention function as partial agonists. Further, when the modulator compounds of the present invention are combined with a corresponding modulator of the other heterodimeric partner, a surprising synergistic enhancement of the activation of the heterodimer pathway can occur. For example, with respect to a RXRα:PPARα heterodimer, the combination of a compound of the present invention with clofibric acid or gemfibrozil unexpectedly leads to a greater than additive (i.e. synergistic) activation of PPARα responsive genes, which in turn is useful to modulate serum cholesterol and triglyceride levels and other conditions associated with lipid metabolism.

Whether acting on an RXR heterodimer pathway, or the RXR homodimer pathway, it will also be understood by those skilled in the art that the dimer-selective RXR modulator compounds of the present invention will prove useful in any therapy in which agonists, partial agonists and/or full antagonists of such pathways will find application. Importantly, because the compounds of the present invention can differentially activate RXR homodimers and RXR heterodimers, their effects will be tissue and/or cell type specific, depending upon the cellular context of the different tissue types in a given patient. For example, compounds of the present invention will exert an RXR antagonist effect in tissues where RXR homodimers prevail, and partial agonist or full agonist activity on the PPAR pathway where RXRα:PPARα heterodimers prevail (e.g., in liver tissue). Thus, the compounds of the present invention will exert a differential effect in various tissues in an analogous fashion to the manner in which various classes of estrogens and antiestrogens (e.g., Estrogen, Tamoxifen, Raloxifen) exert differential effects in different tissue and/or cell types (e.g., bone, breast, uterus). See e.g., M. T. Tzukerman et al., Mol. Endo, 8:21-30 (1994); D. P. McDonnell et al., Mol. Endo., 9:659-669 (1995). However, in the present case, it is believed that the differential effects of the compounds of the present invention are based upon the particular dimer pair through which the compound acts, rather than through different transactiving regions of the estrogen receptor in the case of estrogens and antiestrogens. However, it is possible that they also function, in part, by tissue selectivity.

The particular conditions that may be treated with the compounds of the present invention include, but are not limited to, skin-related diseases, such as actinic keratoses, arsenic keratoses, inflammatory and non-inflammatory acne, psoriasis, ichthyoses and other keratinization and hyperproliferative disorders of the skin, eczema, atopic dermatitis, Darriers disease, lichen planus, prevention and reversal of glucocorticoid damage (steroid atrophy), as a topical anti-microbial, as skin pigmentation agents and to treat and reverse the effects of age and photo damage to the skin. With respect to the modulation of malignant and pre-malignant conditions, the compounds may also prove useful for the prevention and treatment of cancerous and pre-cancerous conditions, including, premalignant and malignant hyperproliferative diseases and cancers of epithelial origin such as cancers of the breast, skin, prostate, cervix, uterus, colon, bladder, esophagus, stomach, lung, larynx, oral cavity, blood and lymphatic system, metaplasias, dysplasias, neoplasias, leukoplakias and papillomas of the mucous mem-branes and in the treatment of Kaposis sarcoma. In addition, the present compounds may be used as agents to treat and prevent various cardiovascular diseases, including, without limitation, diseases associated with lipid metabolism such as dyslipidemias, prevention of restenosis and as an agent to increase the level of circulating tissue plasminogen activator (TPA), metabolic diseases such as obesity and diabetes (i.e., non-insulin dependent diabetes mellitus and insulin dependent diabetes mellitus), the modulation of differentiation and proliferation disorders, as well as the prevention and treatment of neurodegenerative diseases such as Alzheimer's disease, Parkinson's disease and Amyotrophic Lateral Sclerosis (ALS), and in the modulation of apoptosis, including both the induction of apoptosis and inhibition of T-Cell activated apoptosis.

Furthermore, it will be understood by those skilled in the art that the compounds of the present invention, including pharmaceutical compositions and formulations containing these compounds, can be used in a wide variety of combination therapies to treat the conditions and diseases described above. Thus, the compounds of the present invention can be used in combination with modulators of the other heterodimeric partner with RXR (i.e., in combination with PPARα modulators, such as fibrates, in the treatment of cardiovascular disease, and in combination with PPARγ modulators, such thiazolidinediones, in the treatment of diabetes, including non-insulin dependent diabetes mellitus and insulin dependent diabetes mellitus, and with agents used to treat obesity) and with other therapies, including, without limitation, chemotherapeutic agents such as cytostatic and cytotoxic agents, immunological modifiers such as interferons, interleukins, growth hormones and other cytokines, hormone therapies, surgery and radiation therapy.

By utilizing the compounds of the present invention with modulators of the other heterodimeric partner one is able to utilize lower dosages of either or both modulators, thereby leading to a significant decrease in the side-effects associated with such modulators when employed alone at the strengths required to achieve the desired effect. Thus, the modulator compounds of the present invention, when utilized in combination therapies, provide an enhanced therapeutic index (i.e., significantly enhanced efficacy and/or decrease side-effect profiles) over utilization of the compounds by themselves.

Prodrugs are compounds of the present invention, which have chemically or metabolically cleavable groups and become by solvolysis or under physiological conditions the compounds of the invention which are pharmaceutically active in vivo. Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters derived from acidic groups pendent on the compounds of this invention are preferred prodrugs. In some cases it is desirable to prepare double ester type prodrugs such as (acyloxy) alkyl esters or ((alkoxycarbonyl)oxy)alkyl esters. Particularly preferred esters as prodrugs are methyl, ethyl, propyl, isopropyl, n-butyl, isobutyl, tert-butyl, morpholino ethyl, and N,N-diethylglycolamido.

Methyl ester prodrugs may be prepared by reaction of the acid form of a compound of formula I in a medium such as methanol with an acid or base esterification catalyst (e.g., NaOH, H₂SO₄). Ethyl ester prodrugs are prepared in similar fashion using ethanol in place of methanol.

Morpholinylethyl ester prodrugs may be prepared by reaction of the sodium salt of a compound of Structural Formula I (in a medium such as dimethylformamide) with 4-(2-chloroethyl)morphine hydrochloride (available from Aldrich Chemical Co., Milwaukee, Wis. USA, Item No. C4,220-3).

The term “pharmaceutically acceptable” means that the carrier, diluent, excipients and salt must be compatible with the other ingredients of the formulation, and not deleterious to the recipient thereof. Pharmaceutical formulations of the present invention are prepared by procedures known in the art using well known and readily available ingredients.

“Preventing” refers to reducing the likelihood that the recipient will incur or develop any of the pathological conditions described herein.

By virtue of its acidic moiety, a compound of Structural Formula I forms salts with pharmaceutically acceptable bases. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts, zinc salts, and ammonium salts, as well as salts made from physiologically acceptable organic bases such as methylamine, dimethylamine, trimethylamine, ethylamine, diethylamine, triethylamine, morpholine, pyridine, piperidine, piperazine, picoline, nicotinamide, urea, tris(hydroxymethyl)aminomethane, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, N-benzyl-β-phenethylamine, dehydroabietylamine, N,N′-bisdehydroabietylamine, glucamine, N-methylglucamine, collidine, quinine, quinoline, and basic amino acid such as lysine and arginine. These salts may be prepared by methods known to those skilled in the art.

Compounds of Structural Formula I, which are substituted with a basic group, may exist as salts with pharmaceutically acceptable acids. The present invention includes such salts. Examples of such salts include hydrochlorides, hydrobromides, sulfates, methanesulfonates, nitrates, maleates, acetates, citrates, cinnamates, picrate, formate, fumarates, tartrates [e.g. (+)-tartrates, (−)-tartrates or mixtures thereof including racemic mixtures], succinates, benzoates and salts with amino acids such as glutamic acid.

Certain compounds of Structural Formula I and their salts may also exist in the form of solvates, for example hydrates, and the present invention includes each solvate and mixtures thereof.

Certain compounds of Structural Formula I may exist in different tautomeric forms or as different geometric isomers, and the present invention includes each tautomer and/or geometric isomer of compounds of Structural Formula I and mixtures thereof.

Certain compounds of Structural Formula I may exist in different stable conformational forms which may be separable. Torsional asymmetry due to restricted rotation about an asymmetric single bond, for example because of steric hindrance or ring strain, may permit separation of different conformers. The present invention includes each conformational isomer of compounds of Structural Formula I and mixtures thereof.

Certain compounds of Structural Formula I may exist in zwitterionic form and the present invention includes each zwitterionic form of compounds of Structural Formula I and mixtures thereof.

Certain compounds of Structural Formula I and their salts may exist in more than one crystal form. Polymorphs of compounds represented by Structural Formula I form part of this invention and may be prepared by crystallization of a compound of Structural Formula I under different conditions. For example, using different solvents or different solvent mixtures for recrystallization; crystallization at different temperatures; various modes of cooling, ranging from very fast to very slow cooling during crystallization. Polymorphs may also be obtained by heating or melting a compound of Structural Formula I followed by gradual or fast cooling. The presence of polymorphs may be determined by solid probe nmr spectroscopy, ir spectroscopy, differential scanning calorimetry, powder X-ray diffraction or such other techniques.

The language a “therapeutically effective amount” or “pharmaceutically effective amount” is intended to include an amount which is sufficient to mediate a disease or condition and prevent its further progression or ameliorate the symptoms associated with the disease or condition. Such an amount can be administered prophylactically to a patient thought to be susceptible to development of a disease or condition. Such amount when administered prophylactically to a patient can also be effective to prevent or lessen the severity of the mediated condition. Such an amount is intended to include an amount which is sufficient to modulate one or more retinoid X receptor, such as RXR α, RXR β, and/or RXR γ, which mediates a disease or condition. Conditions mediated by retinoid X receptors include diabetes, dermatologic diseases, inflammatory diseases, neurodegenerative diseases, obesity, cardiovascular diseases, cancer and other proliferative diseases, such as atherosclerosis, uterine leiomyomata. In addition, RXR modulators can be used to promote wound healing or to stimulate hair growth.

The compounds of Structural Formula I, and the pharmaceutically acceptable salts, solvates and hydrates thereof, have valuable pharmacological properties and can be used in pharmaceutical preparations containing the compound or pharmaceutically acceptable salts, esters or prodrugs thereof, in combination with a pharmaceutically acceptable carrier or diluent. They are useful as therapeutic substances in preventing or treating diabetes, dermatologic diseases, inflammatory diseases, neurodegenerative diseases, obesity, cardiovascular diseases, cancer, atherosclerosis, uterine leiomyomata, wounds or hair loss in human or non-human animals. Suitable pharmaceutically acceptable carriers include inert solid fillers or diluents and sterile aqueous or organic solutions. The active compound will be present in such pharmaceutical compositions in amounts sufficient to provide the desired dosage amount in the range described herein.

For oral administration, the compound or salts thereof can be combined with a suitable solid or liquid carrier or diluent to form capsules, tablets, pills, powders, syrups, solutions, suspensions and the like.

The tablets, pills, capsules, and the like may also contain a binder such as gum tragacanth, acacias, corn starch or gelatin; excipients such as dicalcium phosphate; a disintegrating agent such as corn starch, potato starch, alginic acid, a lubricant such as magnesium stearate; and a sweetening agent such as sucrose lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained.

The active compounds can also be administered intranasally as, for example, liquid drops or spray.

For parental administration the compounds of the present invention, or salts thereof can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that each syringability exists. It must be stable under the conditions of manufacture and storage and must be preserved against any contamination. The carrier can be solvent or dispersion medium containing, for example, water, ethanol, polyol (e.g. glycerol, propylene glycol and liquid polyethylene glycol), propylene glycol and liquid polyethylene glycol), suitable mixtures thereof, and vegetable oils. The injectable solutions prepared in this manner can then be administered intravenously, intraperitoneally, subcutaneously, or intramuscularly, with intramuscular administration being preferred in humans.

The effective dosage of active ingredient employed may vary depending on the particular compound employed, the mode of administration, the condition being treated and the severity of the condition being treated.

Preferably compounds of the invention or pharmaceutical formulations containing these compounds are in unit dosage form for administration to a mammal. The unit dosage form can be any unit dosage form known in the art including, for example, a capsule, an IV bag, a tablet, or a vial. The quantity of active ingredient (viz., a compound of Structural Formula I or salts thereof) in a unit dose of composition is a therapeutically effective amount and may be varied according to the particular treatment involved. It may be appreciated that it may be necessary to make routine variations to the dosage depending on the age and condition of the patient. The dosage will also depend on the route of administration which may be by a variety of routes including oral, aerosol, rectal, transdermal, subcutaneous, intravenous, intramuscular, intraperitoneal and intranasal.

Pharmaceutical formulations of the invention are prepared by combining (e.g., mixing) a therapeutically effective amount of a compound of the invention together with a pharmaceutically acceptable carrier or diluent. The present pharmaceutical formulations are prepared by known procedures using well known and readily available ingredients.

In making the compositions of the present invention, the active ingredient will usually be admixed with a carrier, or diluted by a carrier, or enclosed within a carrier which may be in the form of a capsule, sachet, paper or other container.

When the carrier serves as a diluent, it may be a solid, lyophilized solid or paste, semi-solid, or liquid material which acts as a vehicle, or can be in the form of tablets, pills, powders, lozenges, elixirs, suspensions, emulsions, solutions, syrups, aerosols (as a solid or in a liquid medium), or ointment, containing, for example, up to 10% by weight of the active compound. The compounds of the present invention are preferably formulated prior to administration.

For the pharmaceutical formulations any suitable carrier known in the art can be used. In such a formulation, the carrier may be a solid, liquid, or mixture of a solid and a liquid. For example, for intravenous injection the compounds of the invention may be dissolved in at a concentration of about 0.05 to about 5.0 mg/ml in a 4% dextrose/0.5% Na citrate aqueous solution.

Solid form formulations include powders, tablets and capsules. A solid carrier can be one or more substance which may also act as flavoring agents, lubricants, solubilisers, suspending agents, binders, tablet disintegrating agents and encapsulating material.

Tablets for oral administration may contain suitable excipients such as calcium carbonate, sodium carbonate, lactose, calcium phosphate, together with disintegrating agents, such as maize, starch, or alginic acid, and/or binding agents, for example, gelatin or acacia, and lubricating agents such as magnesium stearate, stearic acid, or talc.

In powders the carrier is a finely divided solid which is in admixture with the finely divided active ingredient. In tablets the active ingredient is mixed with a carrier having the necessary binding properties in suitable proportions and compacted in the shape and size desired.

Advantageously, compositions containing the compound of Structural Formula I or the salts thereof may be provided in dosage unit form, preferably each dosage unit containing from about 1 to about 500 mg be administered although it will, of course, readily be understood that the amount of the compound or compounds of Structural Formula I actually to be administered will be determined by a physician, in the light of all the relevant circumstances.

Powders and tablets preferably contain from about 1 to about 99 weight lo percent of the active ingredient which is the novel compound of this invention.

Suitable solid carriers are magnesium carbonate, magnesium stearate, talc, sugar, lactose, pectin, dextrin, starch, gelatin, tragacanth, methyl cellulose, sodium carboxymethyl cellulose, low melting waxes, and cocoa butter.

The following pharmaceutical formulations 1 through 8 are illustrative only and are not intended to limit the scope of the invention in any way. “Active Ingredient”, refers to a compound according to Structural Formula I or salts thereof.

### Formulation 1

Hard gelatin capsules are prepared using the following ingredients:

| | Quantity |
|---|---|
| | (mg/capsule) |
| Active Ingredient | 250 |
| Starch, dried | 200 |
| Magnesium stearate | 10 |
| Total | 460 mg |

### Formulation 2

A tablet is prepared using the ingredients below:

| | Quantity |
|---|---|
| | (mg/tablet) |
| Active Ingredient | 250 |
| Cellulose, microcrystalline | 400 |
| Silicon dioxide, fumed | 10 |
| Stearic acid | 5 |
| Total | 665 mg |

The components are blended and compressed to form tablets each weighing 665 mg

### Formulation 3

An aerosol solution is prepared containing the following components:

| | Weight |
|---|---|
| Active Ingredient | 0.25 |
| Ethanol | 25.75 |
| Propellant 22 (Chlorodifluoromethane) | 74.00 |
| Total | 100.00 |

The Active Ingredient is mixed with ethanol and the mixture added to a portion of the propellant 22, cooled to 30° C. and transferred to a filling device. The required amount is then fed to a stainless steel container and diluted with the remainder of the propellant. The valve units are then fitted to the container.

### Formulation 4

Tablets, each containing 60 mg of Active ingredient, are made as follows:

| | |
|---|---|
| Active Ingredient | 60 mg |
| Starch | 45 mg |
| Microcrystalline cellulose | 35 mg |
| Polyvinylpyrrolidone (as 10% solution in water) | 4 mg |
| Sodium carboxymethyl starch | 4.5 mg |
| Magnesium stearate | 0.5 mg |
| Talc | 1 mg |
| Total | 150 mg |

The Active Ingredient, starch and cellulose are passed through a No. 45 mesh U.S. sieve and mixed thoroughly. The aqueous solution containing polyvinylpyrrolidone is mixed with the resultant powder, and the mixture then is passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed on a tablet machine to yield tablets each weighing 150 mg.

### Formulation 5

Capsules, each containing 80 mg of Active Ingredient, are made as follows:

| | |
|---|---|
| Active Ingredient | 80 mg |
| Starch | 59 mg |
| Microcrystalline cellulose | 59 mg |
| Magnesium stearate | 2 mg |
| Total | 200 mg |

The Active Ingredient, cellulose, starch, and magnesium stearate are blended, passed through a No. 45 mesh U.S. sieve, and filled into hard gelatin capsules in 200 mg quantities.

### Formulation 6

Suppositories, each containing 225 mg of Active Ingredient, are made as follows:

| | |
|---|---|
| Active Ingredient | 225 mg |
| Saturated fatty acid glycerides | 2,000 mg |
| Total | 2,225 mg |

The Active Ingredient is passed through a No. 60 mesh U.S. sieve and suspended in the saturated fatty acid glycerides previously melted using the minimum heat necessary. The mixture is then poured into a suppository mold of nominal 2 g capacity and allowed to cool.

### Formulation 7

Suspensions, each containing 50 mg of Active Ingredient per 5 ml dose, are made as follows:

| | |
|---|---|
| Active Ingredient | 50 mg |
| Sodium carboxymethyl cellulose | 50 mg |
| Syrup | 1.25 ml |
| Benzoic acid solution | 0.10 ml |
| Flavor | q.v. |
| Color | q.v. |
| Purified water to total | 5 ml |

The Active Ingredient is passed through a No. 45 mesh U.S. sieve and mixed with the sodium carboxymethyl cellulose and syrup to form a smooth paste. The benzoic acid solution, flavor and color are diluted with a portion of the water and added, with stirring. Sufficient water is then added to produce the required volume.

### Formulation 8

An intravenous formulation maybe prepared as follows:

| | |
|---|---|
| Active Ingredient | 100 mg |
| Isotonic saline | 1,000 ml |

The solution of the above materials generally is administered intravenously to a subject at a rate of 1 ml per minute.

Synthesis

The compounds of the invention can be prepared by reacting a substituted (2-iodo-1-alkylvinyl) azaaryl (VII) and a substituted 5-tributylstannanyl-penta-2,4-dienoic acid alkyl ester (see Scheme III). The substituted (2-iodo-1-alkylvinyl) azaaryl (VII) is prepared from a substituted iodoazaaryl (II) (see Scheme I). The substituted iodoazaaryl (II) is dissolved in a solvent and treated with a catalytic amount of copper iodide and dichlorobis(triphenylphosphine)palladium(II) (typically about 0.05 eq. to about 0.15 eq. of each) and excess aprotic base (typically about 2 eq. to about 10 eq.). After about 5 min. to about 30 min., about 1 eq. to about 3 eq. of trimethylsilyl acetylene (II) is added, and the reaction is heated in a sealed tube to about 50° C. to about 120° C. for about 8 hrs. to about 16 hrs. to form a (substituted azaaryl)-trimethylsilyl acetylene (IV).

The (substituted azaaryl)-trimethylsilyl acetylene (IV) is dissolved in a solvent and treated with about 0.1 eq. to about 0.5 eq. of nickel(II) acetylacetonate (Ni(acac)₂) and about 3 eq. to about 8 eq. of a C₁-C₃ dialkyl zinc (V). Each alkyl group of the C₁-C₃ dialkyl zinc (V) is optionally substituted. Preferably, each alkyl group is substituted with from one to seven halo groups. After about 8 h to about 20 h, an optionally substituted [2-(substituted azaaryl)-2-alkylethen-1-yl]-trimethylsilane (VI) is formed.

A solution of [2-(substituted azaaryl)-2-alkylethen-1-yl]-trimethylsilane (VI) in a nonpolar solvent is cooled to about 10° C. to about −20° C., then about 1 eq. to about 2 eq. of iodine monochloride is added. After about 1 h to about 4 h, a substituted (2-iodo-1-alkylethenyl) azaaryl (VII) is formed.

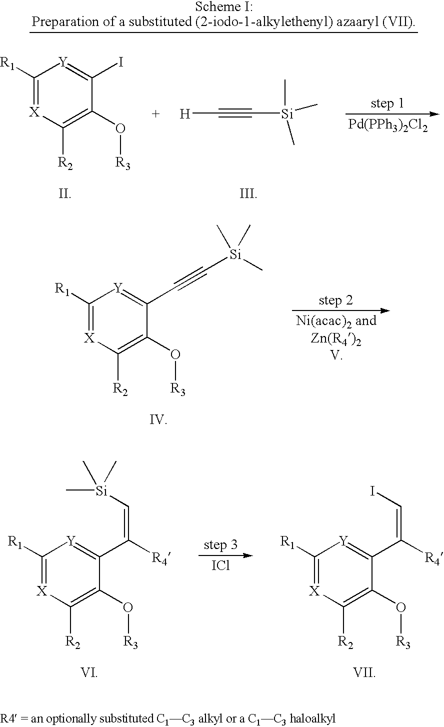

The substituted 5-tributylstannanyl-penta-2,4-dienoic acid alkyl ester (XIII) can be prepared from an optionally substituted alkyl 4-oxocrotonate (XI) (see Scheme II). In the first step, dialkylchlorophosphate (IX) and lithium hexamethyldisilazane (LiHMDS) are added to a solution of methyl phenyl sulfone (VIII) that is optionally substituted with a fluoro group in an aprotic solvent, preferably an ether, that has been cooled to about −50° C. to about −100° C. After about 15 min. to about 1 hr., the optionally substituted alkyl 4-oxocrotonate (XI) is added, and the reaction is allowed to warm to room temperature and is stirred for about 8 hrs. to about 20 hrs. to form an optionally substituted 5-benzenesulfonyl-penta-2,4-dienoic acid alkyl ester (XII). About 1.5 eq. to 2.5 eq. of the methyl phenyl sulfone (VI), about 1.5 eq. to about 2.5 eq. of the dialkylchlorophosphate (IX), and about 3.0 eq. to about 5 eq. of the lithium hexamethyldisilazane with respect to the alkyl 4-oxocrotonate (XI) are typically present in the reaction mixture.

A mixture of the 5-benzenesulfonyl-penta-2,4-dienoic acid alkyl ester (XII), about 1.5 eq. to about 3 eq. of tributyl tin hydride (SnBu₃H) and a catalytic amount of a free radical initiator such as 2,2′-azobisisobutyronitrile (AIBN) in an organic solvent is heated to about 50° C. to about 120° C. for about 8 hrs. to about 20 hrs. to form an optionally substituted 5-tributylstannayl-penta-2,4-dienoic acid alkyl ester (XIII).

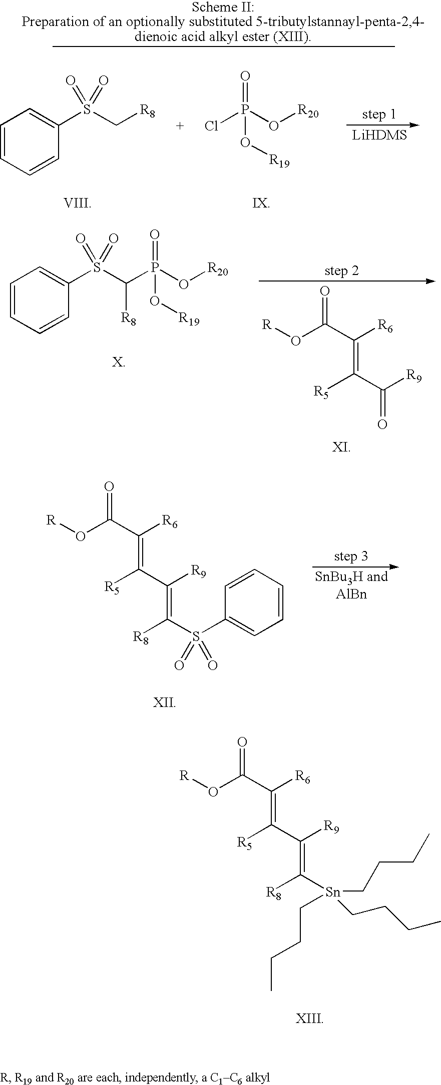

The substituted (2-iodo-1-alkylethenyl) azaaryl (VII) and the 5-tributylstannayl-penta-2,4-dienoic acid alkyl ester (XI) (about 1 eq. to about 1.5 eq.) are combined in an organic solvent with a catalytic amount (about 0.05 eq. to about 0.15 eq.) of dichlorobis(triphenylphosphine)palladium(II). The reaction is heated to about 50° C. to about 100° C. for about 1 h to about 4 h to form an optionally substituted 7-(substituted azaaryl)-hepta-2,4,6-trienoic acid alkyl ester (XIV). A 7-(substituted azaaryl)-hepta-2,4,6-trienoic acid (XV) can be formed by treating the 7-(substituted azaaryl)-hepta-2,4,6-trienoic acid alkyl ester (XIV) with an alkali metal hydroxide (see Scheme III).

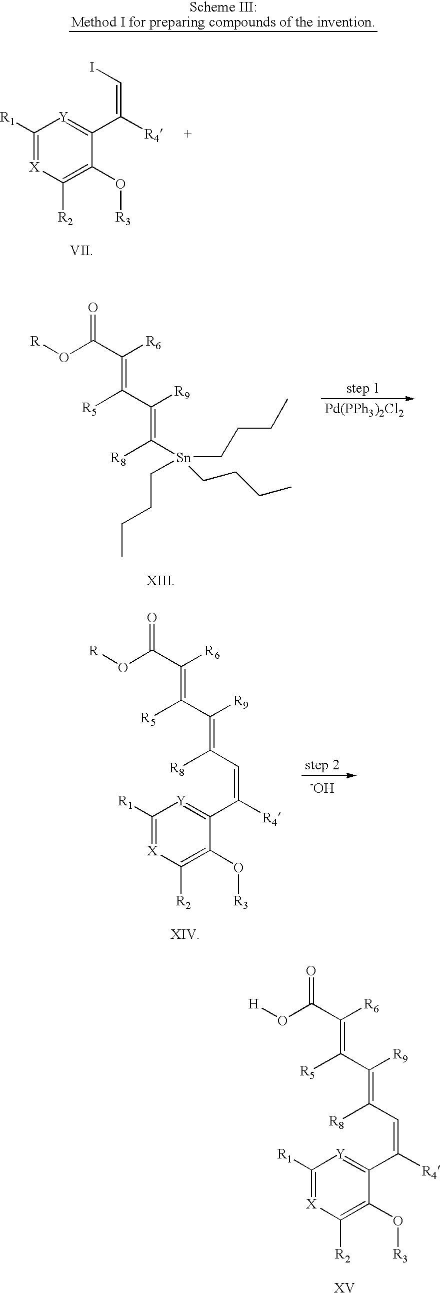

Alternativly, compounds of the invention can be prepared by a second method from a azaaryl substituted with α,β-unsaturated carbonyl (XVI) (see Scheme IV). In this method, compound X is prepared via the method of Scheme II, step 1. A azaaryl substituted with α,β-unsaturated carbonyl (XVI) is added to a solution of compound X in an aprotic solvent maintained at about −50° C. to about −100° C. The reaction is allowed to warm to room temperature and is stirred for about 8 h to about 20 h to form an optionally substituted 1-benzenesulfonyl-4-(substituted azaaryl)-buta-1,3-diene (XVII). About 1.5 to 2.5 eq. of the methyl phenyl sulfone (VIII) which is optionally substituted with a fluoro group, about 1.5 eq. to about 2.5 eq. of the dialkylchlorophosphate (IX), and about 3.0 eq. to about 5 eq. of the lithium hexamethyldisilazane with respect to compound XVI are typically present in the reaction mixture.

A mixture of the 1-benzenesulfonyl-4-(substituted azaaryl)-buta-1,3-diene (XVII), about 1.5 eq. to about 3 eq. of tributyl tin hydride (SnBu₃H) and a catalytic amount of a free radical initiator, such as AIBN, in an organic solvent is heated to about 50° C. to about 120° C. for about 8 h to about 20 h to form an optionally substituted 1-tributylstannayl-4-(substituted azaaryl)-buta-1,3-diene (XVIII).

A mixture of the 1-tributylstannayl-4-(substituted azaaryl)-buta-1,3-diene (XVIII), about 1 eq. to about 2 eq. of an optionally substituted 3-iodo-pro-2-enoic acid (XIX) and about 0.05 eq. to about 0.15 eq. of dichlorobis(triphenylphosphine)-palladium(II) (also referred to herein al “Pd(PPh₃)₂Cl₂”) was heated to about 50° C. to about 100° C. for about 1 h to about 4 h. The reaction is then poured into a potassium fluoride solution and stirred at room temperature for about 0.5 hrs. to about 2 hrs. to form an optionally substituted 7-(substituted azaaryl)-hepta-2,4,6-trienoic acid (XX).

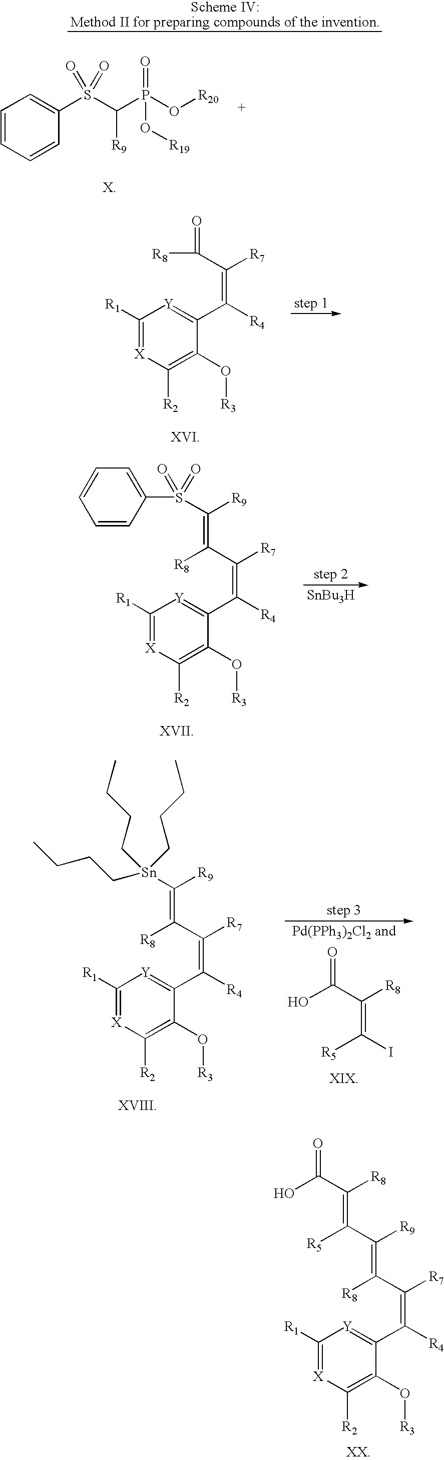

Compounds of the invention can be synthesized by a third method in which an azaaryl substituted with an α,β-unsaturated carbonyl (XVI) undergoes an aldol condensation with a ketone (XXI) followed by an elimination reaction to form an optionally substituted 5-(substituted azaaryl)-1-oxopenta-2,4-diene (XXII). The reaction is carried out in a basic solvent such as piperidine or pyridine in the presence of about 1 eq. to about 1.5 eq. of an acid. The ketone (XXI) is typically present in a large excess. The 5-(substituted azaaryl)-1-oxopenta-2,4-diene (XXII) forms after stirring the reaction mixture for about 0.5 h to about 2 h at room temperature.

A solution of an optionally substituted trialkyl phosphonoacetate (XXIII) in an aprotic solvent is treated with about 1 eq. to about 1.5 eq. of sodium hydride at room temperature. After about 0.5 hrs. to about 1.5 hrs., about 0.5 eq. to about 1 eq. of the 5-(substituted azaaryl)-1-oxopenta-2,4-diene (XXI) is added to a solution, and the reaction is stirred for about 8 h to about 20 h to form 7-(substituted azaaryl)-hepta-2,4,6-trienoic acid alkyl ester (XXIV) (see Scheme V). A 7-(substituted azaaryl)-hepta-2,4,6-trienoic acid (XX) can be formed by treating the 7-(substituted azaaryl)-hepta-2,4,6-trienoic acid all ester (XXIV) with an alkali metal hydroxide as in Scheme III, step 2.

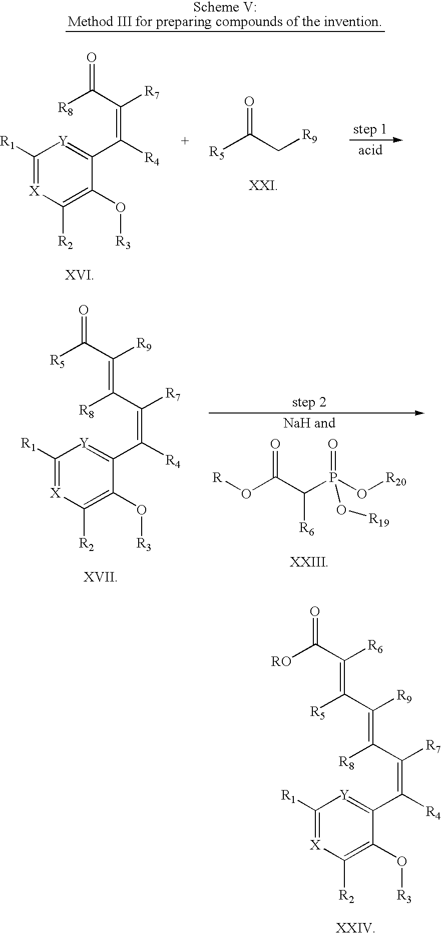

Alternatively, compounds of the invention can be prepared by reacting an azaaryl substituted with an α,β-unsaturated carbonyl (XVI) with an anion of a trialkylphosphono acetate (XXXIX) (see Scheme VI). In this method, a solution of trialkyl phosphono acetate (XXIX) in an aprotic solvent at about −25° C. to about 10° C. is treated with about 1 eq. to about 1.5 eq. of sodium hydride. After about 0.5 h to about 1.5 h, the azaaryl substituted with an α,β-unsaturated carbonyl (XVI) is added, and the mixture is stirred for about 4 h to about 24 h to form an optionally substituted 5-(substituted azaaryl)-penta-2,4-dienoic acid alkyl ester (XL).

The 5-(substituted azaaryl)-penta-2,4-dienoic acid alkyl ester (XL) is treated with a reducing agent, such as sodium borohydride, lithium aluminum hydride or diisobutylaluminum hydride, to form an optionally substituted 5-(substituted azaaryl)-penta-2,4-dien-1-ol (XLI). The reaction is typically carried out in a polar solvent at about −25° C. to about 10° C. About 2 eq. to about 5 eq. of the reducing agent is used with respect to the 5-(substituted azaaryl)-penta-2,4-dienoic acid alkyl ester (XL). Typically, the reaction is followed by thin layer chromatography (TLC) to determine when the reaction is complete.

The allylic hydroxy group of 5-(substituted azaaryl)-penta-2,4-dien-1-ol (XLI) is converted to an aldehyde to form an optionally substituted 5-(substituted azaaryl)-penta-2,4-dien-1-al (XLII) by treatment with about 1 eq. to about 2 eq. of 4-methylmorpholine N-oxide (hereinafter “NMO”) and a cataylic amount of tetrapropylammonium perruthenate (hereinafter “TPAP”) (about 0.01 eq. to about 0.1 eq.). The reaction is carried out in a nonpolar solvent at room temperature.

Alternatively, the allylic hydroxy can be oxidized to an aldehyde to form an optionally substituted 5-(substituted azaaryl)-penta-2,4-dien-1-al (XLII) by treatment of 5-(substituted azaaryl)-penta-2,4-dien-1-ol (XLI) with about 1 eq. to about 2 eq. of Dess-Martin periodinane. This reaction is carried out at room temperature and is complete in about 2 h to about 8 h. When the reaction is complete, it is diluted with an organic solvent that is not miscible with water and washed with an aqueous NaOH solution.

When R₅ is an optionally substituted C₁-C₃ alkyl or a C₁-C₃ haloalkyl, steps 4 and 5 of Scheme VI are carried out to form a 1-alkyl-5-(substituted azaaryl)-1-oxopenta-2,4-diene (XXII) which can be treated as in Scheme V, step 2 to form an optionally substituted 7-(substituted azaaryl)-hepta-2,4,6-trienoic acid alkyl ester (XXIV). When R₅ is a hydrogen, 5-(substituted azaaryl)-penta-2,4-dien-1-al (XLII) can be treated as in Scheme V, step 2 to form an optionally substituted 7-(substituted azaaryl)-hepta-2,4,6-trienoic acid alkyl ester (XXIV).

In step 4 of Scheme VI, about 1 eq. to about 2 eq. of a Grignard reagent (XLIII) is added to a solution of 5-(substituted azaaryl)-penta-2,4-dien-1-al (XLII) in a polar aprotic solvent that is maintained at about −25° C. to about 10° C. The solution is stired for about 1 h to about 6 h to form a 1-alkyl-5-(substituted azaaryl)-penta-2,4-dien-1-ol (XLIV).

The allylic alcohol of 1-alkyl-5-(substituted azaaryl)-penta-2,4-dien-1-ol (XLIV) can be oxidized to a ketone by treating it with NMO and TRAP or with Dess-Martin periodinane as described above to form a 1-alkyl-5-(substituted azaaryl)-1-oxopenta-2,4-diene (XXII).

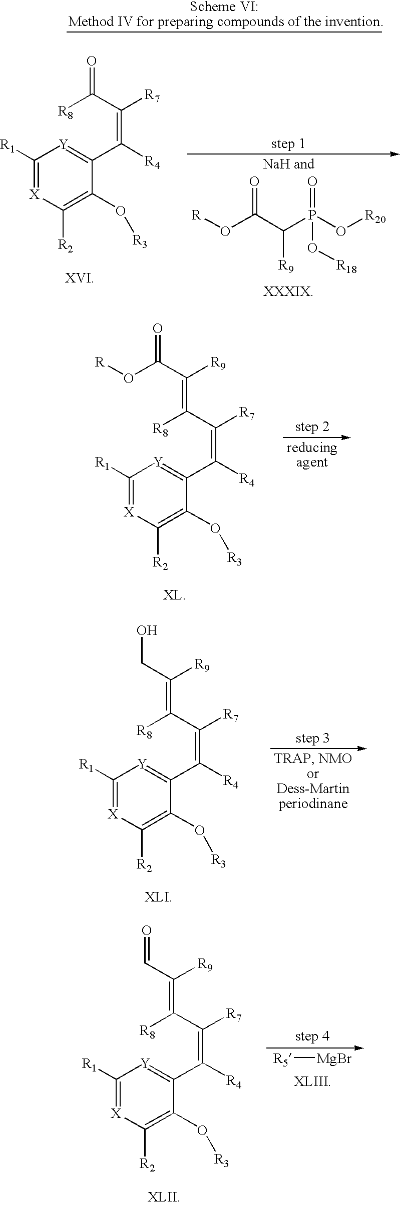

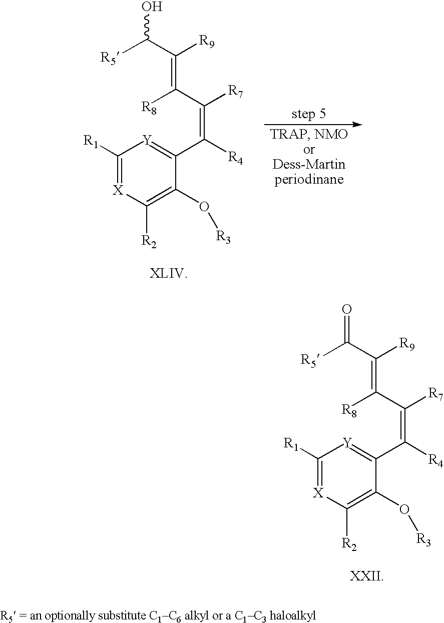

Compounds of the invention can also be prepared from an acetyl-hydroxyazaaryl (XXVII) (see Schemes IX and XI). The acyl-hydroxyazaaryl (XXVII) can be prepared by cooling a solution of halo-hydroxyazaaryl (XXV) in an aprotic solvent to about −50° C. to about −100° C. then adding about 1 eq. to about 2.5 eq. of an alkyl lithium compound, such as n-butyl lithium, iso-butyl lithium or tert-butyl lithium. After about 15 min. to about 1 h, the solution is warmed to room temperature and stirred for about 1 h to about 4 h. The solution is then cooled to about −50° C. to about −100° C., and an excess of an alkyl ester (XXVI) that is optionally substituted with from one to three fluoro groups is added. The solution is then allowed to warm to about −20° C. to about 10° C. and stirred for about 15 min. to about 2 h to afford the optionally substituted acyl-hydroxyazaaryl (XXVII) (see Scheme VII).

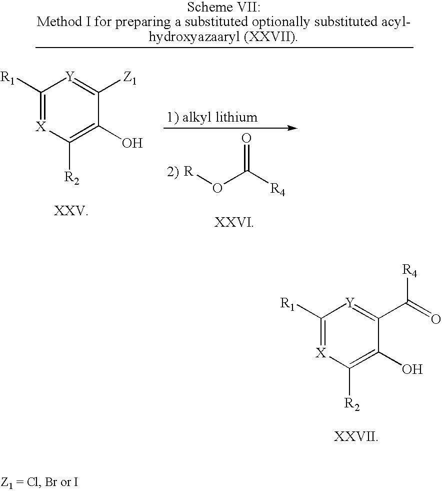

Alternatively, the optionally substituted acyl-hydroxyazaaryl can be prepared by the method depicted in Scheme VIII. In this method, an optionally substituted hydroxyazaaryl (XLV) is treated with a halide (L) in the presence of sodium carbonate. Typically, about 1 eq. to about 2 eq. of a halide (L) is added to a mixture of the acyl-hydroxyazaaryl and sodium carbonate in water or water and a water miscible organic solvent which is maintained at about 50° C. to about 100° C. The reaction is complete in about 15 min. to about 1 h to form an optionally substituted halo-hydroxyazaaryl (XXV).

The halo-hydroxyazaaryl (XXV) is protected with a aromatic hydroxy protecting group to form a protected halo-hydroxyazaaryl (XLVII). The protected halo-hydroxyazaaryl (XLVII) is mixed with about 1 eq. to about 2 eq. of a tributyl-(1-alkoxy-vinyl)-staanane (XLVIII) in an organic solvent in the presence of about 0.05 eq. to about 0.1 eq. of Pd(PPh₃)₂Cl₂. The reaction is sparged with an inert gas, such as N₂ or Ar, to remove oxygen, then heated to about 50° C. to about 100° C. under an inert atmosphere for about 8 h to about 24 h to form a protected optionally substituted acyl-hydroxyazaaryl (XLIX). The protected acyl-hydroxyazaaryl (XLIX) can be deprotected to form an acyl-hydroxyazaaryl (XXVII).

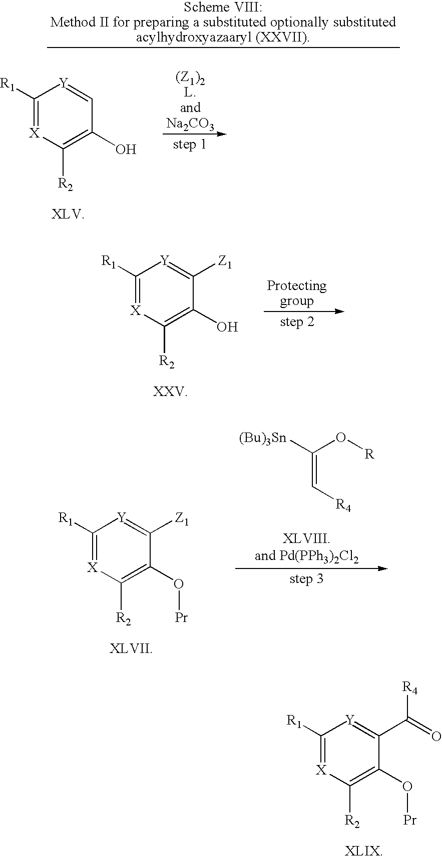

7-(substituted azaaryl)-hepta-2,4,6-trienes in which R₄ and R₇ are in a cis configuration can be prepared from an optionally substituted acyl-hydroxyazaaryl (XXVII) using the method depicted in Scheme IX. In this method, a solution of a (carbalkoxymethylene) triphenylphosphorane (XXVIII) and a 2-acyl-hydroxyazaaryl (XXVII) in an aprotic solvent is heated to about 80° C. to about 120° C. for about 3 days to about 7 days to form a substituted azacoumarin (XXIX).

The substituted azacoumaxin (XXIX) is treated with a reducing agent, such as sodium borohydride, lithium aluminum hydride or diisobutylaluminum hydride, to form a substituted 3-hydroxy-azaaryl)-prop-2-en-1-ol (XXX). The reaction is typically carried out in a polar solvent at about −25° C. to about 10° C. About 2 eq. to about 5 eq. of the reducing agent is used with respect to the azacoumarin (XXIX). Typically, the reaction is followed by thin layer chromatography (TLC) to determine when the reaction is complete.

The aromatic hydroxy group is alkylated to form an optionally substituted 3-(alkoxy-azaaryl)-prop-2-en-1-ol (XXXII) by treating the substituted 3-(hydroxy-azaaryl)-prop-2-en-1-ol (XXX) in the presense of cesium fluoride or cesium carbonate with an optionally substituted aliphatic halide (R₃-Z₁ represents an optionally substituted C₁-C₉ alkyl halide, an optionally substituted C₃-C₇ cycloalkyl halide or an optionally substituted aralkyl in which the alkyl portion is substituted with a halide. Collectively, they are referred to herein as “an aliphatic halide” (XXXI)). The reaction is carried out in a polar solvent at ambient temperatures. The aliphatic halide (XXXI) is present in about 1.1 eq. to about 2 eq. with respect to the 3-(hydroxy-azaaryl)-prop-2-en-1-ol (XXX) and the cesium fluoride or cesium carbonate is present in about 1.5 eq. to about 3 eq. Typically, the reaction is followed by TLC to determine when the reaction is complete.

The allylic hydroxy group of 3-(alkoxy-azaaryl)-prop-2-en-1-ol (XXXII) is converted to an aldehyde to form an optionally substituted 3-(alkoxy-azaaryl)-prop-2-en-1-al (XXXIII) by treatment with about 1 eq. to about 2 eq. of NMO and a cataylic amount of TPAP or with a Dess-Martin periodinane as described above for step 3 of Scheme VI.

An anion of an optionally substituted trialkyl 3-phosphocrotonate (XXXIV) is formed by treating the trialkyl phosphocrotonate (V) in a solution of a polar aprotic solvent maintained at about −50° C. to about −100° C. with about 1 eq. to about 1.5 eq. of an alkyl lithium. After addition of the alkyl lithium, the mixture is stirred for about 10 min. to about 30 min., then 3-(alkoxy-azaaryl)-prop-2-en-1-al (XXXIII) is added to the mixture. The solution is allowed to warm up to room temperature to form an optionally substituted 7-(substituted azaaryl)-hepta-2,4,6-trienoic acid alkyl ester (XXXV) in which R₄ and R₇ are in a cis configuration. The 7-(substituted azaaryl)-hepta-2,4,6-trienoic acid alkyl ester (XXXV) can be treated with an alkali hydroxide as in Scheme III, step 2 to form an optionally substituted 7-(substituted azaaryl)-hepta-2,4,6-trienoic acid (XX).

Examples 1 through 5 were prepared using the methods depicted in Schemes VIII and IX.

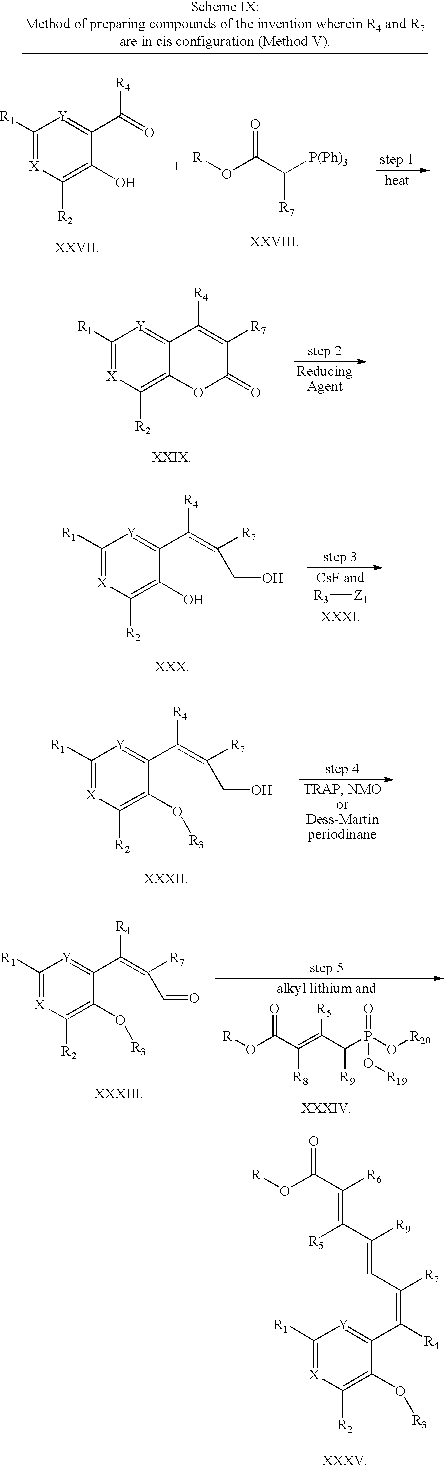

Alternatively, the substituted azacoumarin (XXIX) can be formed from a trialkyl phosphonoacetate (LI) (see Scheme X). In this method, a solution of trialkyl phosphonoacetate (LI) in an aprotic solvent at about −25° C. to about 10° C. is treated with about 1 eq. to about 1.5 eq. of sodium hydride. After about 0.5 h to about 1.5 h, the optionally substituted acyl-hydroxyazaaryl (XXVII) is added and the mixture is stirred for about 4 h to about 24 h to form a substituted azacoumarin (XXIX).

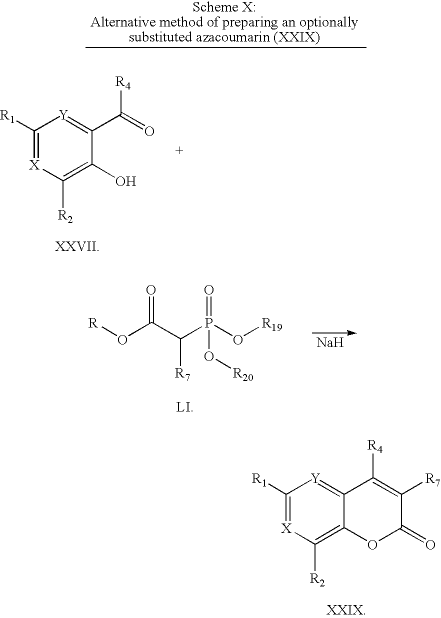

To prepare compounds of the invention in which R₄ and R₇ are in the trans configuration (see Scheme XI), an optionally substituted acyl-hydroxyazaaryl (XXVII) in a polar aprotic solvent maintained at about −25° C. to about 10° C. is treated with about 1 eq. to about 1.5 eq. of sodium hydride to form an anion. About 1 eq. to about 2 eq. of an optionally substituted aliphatic halide (XXXI) is added to the mixture. The reaction is allowed to warm up to room temperature and stirred for about 24 h to about 72 h more to form an optionally substituted acyl-alkoxyazaaryl (XXXVI).

An anion of a trialkyl phosphonoacetate (XXVIII) is formed by treating a trialkyl phosphonoacetate (XXXVI) in a solution of an aprotic solvent maintained at about −25° C. to about 10° C. with about 1 eq. to about 1.5 eq. of sodium hydride. After about 0.5 h to about 1.5 h, the optionally substituted acyl-alkoxyazaaryl (XXXVI) is added, and the mixture is allowed to warm to room temperature and stirred for about 8 h to about 24 h to form an optionally substituted 3-(alkoxy-azaaryl)-prop-2-enoic acid alkyl ester (XXXVII) as a mixture of isomers in which the major product is an isomer wherein R₄ and R₇ are in the trans configuration.

The 3-(alkoxy-azaaryl)-prop-2-enoic acid alkyl ester (XXXVII) is treated with a reducing agent, such as sodium borohydride, lithium aluminum hydride or diisobutylaluminum hydride, to form an optionally substituted 3-(alkoxy-azaaryl)-prop-2-en-1-ol. The reaction is typically carried out in a polar solvent at about −25° C. to about 10° C. About 2 eq. to about 5 eq. of the reducing agent is used with respect to the 3-(alkoxy-azaaryl)-prop-2-enoic acid alkyl ester (XXII) Typically, the reaction is followed by thin layer chromatography (TLC) to determine when the reaction is complete.

The 3-(alkoxy-azaaryl)-prop-2-en-1-ol (XXXIII) can be treated as in Scheme IX, steps 4 and 5 to form an optionally substituted 7-(substituted azaaryl)-hepta-2,4,6-trienoic acid alkyl ester (XXXV) in which R₄ and R₇ are in a trans configuration. The 7-(substituted azaaryl)-hepta-2,4,6-trienoic acid alkyl ester (XXXV) can be treated with an alkali hydroxide as in Scheme III, step 2 to form an optionally substituted 7-(substituted azaaryl)-hepta-2,4,6-trienoic acid (XX).

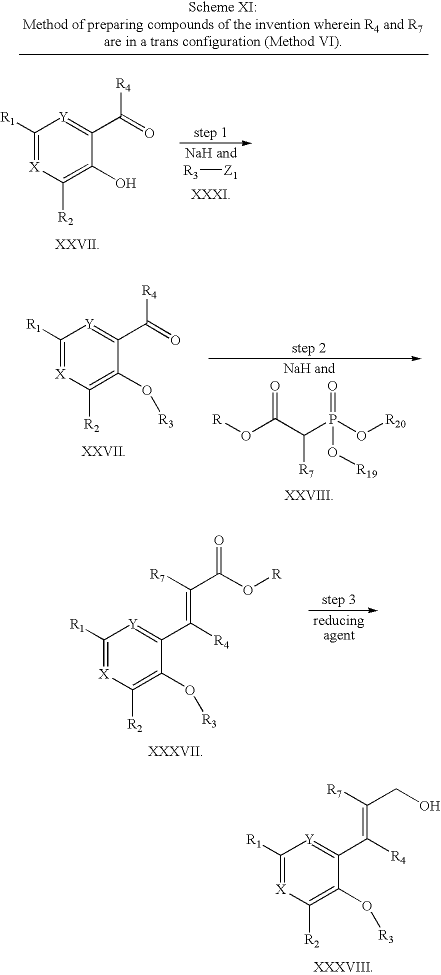

Methods of converting a 7-(substituted azaaryl)-hepta-2,4,6-trienoic acid or a 7-(substituted azaaryl)-hepta-2,4,6-trienoic acid alkyl ester to an anhydride are known to those skilled in the art. For example, a 7-(substituted azaaryl)-hepta-2,4,6-trienoic acid can be converted to an anhydride via an exchange reaction with an ester (see March, Advanced Organic Chemistry, 3^{rd} Edition (1985), John Wiley & Sons, pages 355-356, the entire teachings of which are encorporated herein by reference).

Methods of converting a 7-(substituted azaaryl)-hepta-2,4,6-trienoic acid alkyl ester to an amide are also known to those skilled in the art. For example, a 7-(substituted azaaryl)-hepta-2,4,6-trienoic acid alkyl ester can be converted to an amide by reacting it with ammonia or a primary or secondary amine (see March, Advanced Organic Chemistry, 3^{rd} Edition (1985), John Wiley & Sons, page 375, the entire teachings of which are encorporated herein by reference).

### EXAMPLES

General. All reagents were obtained from commercial suppliers and used without further purification. Solvents were obtained anhydrous from commercial suppliers and used without further purification. All organic solutions were routinely dried over magnesium sulfate (MgSO₄) or sodium sulfate (Na₂SO₄) and solvents were removed under vacuum using a rotary evaporator. ¹H spectra were recorded on a Varian 400 at 400 MHz while ¹³C NMR spectra were recorded on a Bruker Avance 250 (or Avance 300) at 63 MHz (or 75 MHz) as noted. Spectra were obtained using CDCl₃ unless otherwise noted. Chemical shifts are reported in ppm (3) and coupling constants (J) are reported in Hertz. Flash chromatography was performed on an Isco Sg100C separation system using Isco prepacked columns. Melting points were measured on a Gallenkamp melting point apparatus and are uncorrected.

### Example 1

### 7-(3-Butoxy-2,6-diisopropyl-pyridin-4-yl)-3-methyl-octa-2(E),4(E),6(Z)-trienoic Acid

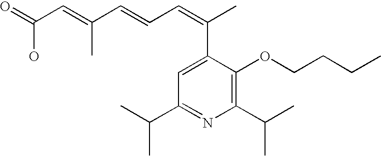

A. 2,6-Diiodo-3-methoxymethoxy-pyridine

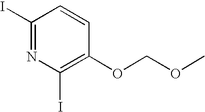

To a 0° C. solution of 2,6-diiodo-3-hydroxy-pyridine (5.01 g, 14.4 mmol) in DMF (25 mL) was added chloromethyl-methylether (1.30 mL, 17.1 mmol), then sodium hydride (720 mg, 18.0 mmol). The solution was warmed to room temperature and stirred for 3 h. The solution was quenched with saturated NaHCO₃ (50 mL) and extracted with ether (3×50 mL). The organic layers were combined, washed with H₂O (50 mL) and brine (50 mL), then dried, filtered, and concentrated. The crude material was purified by flash chromatography (10% to 30% EtOAc/hexanes) to give 2,6-diiodo-3-methoxymethoxy-pyridine (5.45 g, 97%) as a white solid. ¹H NMR (400 MHz): δ 7.51 (d, 1H, J=8.3), 6.95 (d, 1H, J=8.3), 5.22 (s, 2H), 3.48 (s, 3H). MS [EI+] 391.9 (M+H)⁺.

B. 2,6-Diisopropenyl-pyridin-3-ol

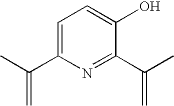

To a solution of 2,6-diiodo-3-methoxymethoxy-pyridine (10.5 g, 26.8 mmol) in DMF (200 mL) was added tributyl-isopropenylstannane (20.4 g, 61.6 mmol), potassium carbonate (7.42 g, 53.7 mmol), and dichlorobis(triphenyl-phosphine) palladium(II) (1.75 g, 2.49 mmol). The mixture was sparged with N₂ then heated to 110° C. for 2 h. The black mixture was cooled to room temperature and quenched with a 5 M solution of aqueous KF (100 mL). The mixture was stirred for 1 h then extracted with ether (3×200 mL). The organic layers were combined, washed with H₂O (2×100 mL) and brine (100 mL), then dried, filtered, and concentrated. The crude material was partially purified by flash chromatography twice (0 to 10% ethyl acetate/hexanes) to give 2,6-diisopropenyl-3-methoxymethoxy-pyridine which was used directly in the next reaction.

To a solution of the partially purified 2,6-diisopropenyl-3-methoxymethoxy-pyridine in THF (100 mL) was added 5 N HCl solution (20 mL). The solution was warmed to 50° C. and stirred for 2 h. The solution was then cooled to room temperature, quenched with saturated NaHCO₃ (75 mL), and extracted with ethyl acetate (2×50 mL). The organic layers were combined and washed with brine (100 mL) then dried, filtered, and concentrated. The crude material was purified by flash chromatography (20% ethyl acetate/hexanes) to give 2,6-diisopropenyl-pyridin-3-ol (3.08 g, 66% for 2 steps) as a white solid. ¹H NMR (400 MHz): δ 7.29 (d, 1H, J=8.6), 7.18 (d, 1H, J=8.6), 5.73 (m, 1H), 5.54 (m, 1H), 5.35 (m, 1H), 5.16 (m, 1H), 2.22 (m, 3H), 2.16 (m, 3H). MS [EI+] 176.2 (M+H)⁺, [EI−] 174.3 (M−H)⁻.

C. 4-Iodo-2,6-diisopropyl-pyridin-3-ol

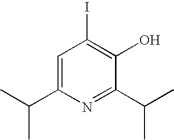

To a solution of 2,6-diisopropenyl-pyridin-3-ol in 1:1 tetrahydrofuran:ethanol (200 mL) was added 5% Pd/C (1.4 g). The mixture was heated to 40° C. in a Parr shaker under H₂ atm (60 psi) for 6 h. The mixture was filtered and concentrated then analyzed by mass spectrometry which showed that only half of the material was completely reduced. The material was re-dissolved in 1:1 tetrahydrofuran: ethanol (200 mL) and 5% Pd/C (1.4 g) was added. The mixture was heated to 40° C. in a Parr shaker under H₂ atm (60 psi) for another 6 h. After filtration, analysis of the filtrate by mass spectrometry showed that complete reduction had occurred. The filtrate was concentrated, and the crude material was purified by flash chromatography to give 2,6-diisopropyl-pyridin-3-ol (2.40 g, 84%) as a white solid.

¹H NMR (400 MHz): δ 6.95 (d, 1H, J=7.8), 6.84 (d, 1H, J=8.3), 3.28 (sept., 1H, J=6.8), 2.97 (sept., 1H, J=6.8), 1.27 (d, 6H, J=6.8), 1.23 (d, 6H, J=67.3). MS [EI+] 180.2 (M+H)⁺, [EI−] 178.3 (M−H)⁻.

To a 70° C. solution of 2,6-diisopropyl-pyridin-3-ol (1.02 g, 5.67 mmol) and sodium carbonate (1.82 g, 17.2 mmol) in a mixture of 3:2H₂O:dimethylsulfoxide (97 mL) was added I₂ (1.73 g, 6.82 mmol). The mixture was stirred for 25 min., then cooled to room temperature, diluted with H₂O, and extracted with diethyl ether (3×75 mL). The organic layers were combined, washed with saturated Na₂SO₃ (50 mL), H₂O (50 mL) and brine (50 mL), then dried, filtered, and concentrated. The crude material was purified by flash chromatography (5% to 10% ethyl acetate/hexanes) to give 4-iodo-2,6-diisopropyl-pyridin-3-ol (1.60 g, 92%) as an orange oil. ¹H NMR (400 MHz): δ 7.22(s, 1H), 5.05 (br s, 1H), 3.38 (sept., 1H, J=6.8), 2.90 (sept., 1H, J=6.8), 1.23 (d, 6H, J=7.3), 1.21 (d, 6H, J=6.8).

D. 1-(3-Hydroxy-2,6-diisopropyl-pyridin-4-yl)-ethanone

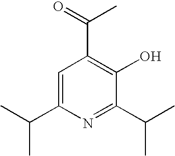

To a 0° C. solution of 4-iodo-2,6-diisopropyl-pyridin-3-ol (4.50 g, 14.7 mmol) in dimethylformamide (100 mL) was added chloromethyl-methylether (1.45 mL, 19.1 mmol) and sodium hydride (730 mg, 18.2 mmol). The mixture was stirred at 0° C. for 15 min., then at room temperature for 2 h. The mixture was quenched with saturated NaHCO₃ (50 mL) and extracted with diethyl ether (3×75 mL). The organic layers were combined, washed with H₂O (50 mL) and brine (50 mL), then dried, filtered, and concentrated. The crude material was purified by flash chromatography (0 to 5% ethyl acetate/hexanes) to give 4-iodo-2,6-diisopropyl-3-methoxymethoxy-pyridine (4.81 g, 94%) as a light yellow oil. ¹H NMR (400 MHz): δ 7.37 (s, 1H), 5.00 (s, 2H), 3.65 (s, 3H), 3.44 (sept., 1H, J=6.8), 2.92 (sept., 1H, J=6.8), 1.22 (d, 6H, J=6.8), 1.21 (d, 6H, J=6.8). MS [EI+] 350.2 (M+H)⁺.

To a solution of 4-iodo-2,6-diisopropyl-3-methoxymethoxy-pyridine (4.79 g, 13.7 mmol) in dimethylformamide (80 mL) was added tributyl-(1-ethoxy-vinyl)-stannane (6.45 g, 17.9) and dichlorobis(triphenylphosphine) palladium(1) (972 mg, 1.38 mmol). The mixture was sparged with N₂ then heated to 80° C. for 17 h under N₂ atm. The mixture was cooled to room temperature and quenched with a solution of 1.9 N aqueous KF (20 mL). After stirring for 1 h, the mixture was filtered, and the filtrate was extracted with diethyl ether (3×75 mL). The organic layers were combined, washed with H₂O (2×75 mL) and brine (75 mL), then dried, filtered, and concentrated. The crude material was partially purified by flash chromatography (10% ethyl acetate/hexanes) to give 4-(1-ethoxy-vinyl)-2,6-diisopropyl-3-methoxymethoxy-pyridine as a light yellow oil which contained significant impurites. The mixture was subjected directly to the next reaction without further purification.

The above material was dissolved in acetone (60 mL) and treated with SN HCl (16 mL). The solution was stirred at room temperature for 18 h then concentrated to half volume. The solution was neutralized with saturated NaHCO₃ and extracted with ethyl acetate (2×50 mL). The organic layers were combined and washed with brine (100 mL) then dried, filtered, and concentrated. The crude material was purified by flash chromatography (5% ethyl acetate/hexanes) to give 1-(3-hydroxy-2,6-diisopropyl-pyridin-4-yl)-ethanone (2.45 g, 81% 2 steps) as a light yellow oil. ¹H NMR (400 MHz): δ 11.7 (s, 1M), 7.11 (s, 1H), 3.51 (sept., 1H, J=6.8), 2.99 (sept., 1H, J=6.8), 2.63 (s, 3H), 1.26 (d, 6H, J=6.8), 1.24 (d, 6H, J=7.3). MS [EI+] 222.1 (M+H)⁺, [EI−] 220.1 (M−H)⁻.

E. 4-(3-Hydroxy-1-methyl-(Z)propenyl)-2,6-diisopropyl-pyridin-3-ol

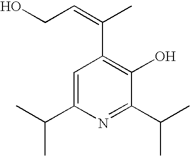

To a solution of 1-(3-hydroxy-2,6-diisopropyl-pyridin-4-yl)-ethanone (2.20 g, 9.94 mmol) in toluene (50 mL) was added (carbethoxymethylene)triphenylphosphorane (4.13 g, 11.9 mmol). The solution was heated to reflux and stirred for 5 days. The solution was cooled to room temperature and concentrated. The crude material was purified by flash chromatography (0 to 15% ethyl acetate/hexanes) to give 6,8-diisopropyl-4-methyl-pyrano[2,3-c]pyridin-2-one (2.02 g, 83%) as a light yellow oil that solidified upon standing. ¹H NMR (250 MHz): δ 7.05 (s, 1H), 6.40 (d, 1H, J=1.2), 3.69 (sept., 1H, J=6.8) 3.03 (sept., 1H, J=6.9), 2.40 (d, 3H, J=1.2), 1.28 (d, 12H, J=6.8). ¹³C NMR (75 MHz): δ 161.4, 159.8, 155.2, 151.5, 143.9, 125.0, 118.7, 111.3, 35.9, 29.0, 22.7(2), 21.3(2), 18.4. MS [EI+] 246.1 (M+H)⁺, [EI−] 244.2 (M−H)⁻.

To a solution of 6,8-diisopropyl-4-methyl-pyrano[2,3-c]pyridin-2-one (1.97 g, 8.03 mmol) in ether (45 mL) at −78° C. was added a 1 M solution of lithium aluminum hydride in ether (8.0 mL, 8.0 mmol). The solution was stirred at −78° C. for 1 h, then warmed to 0° C. and stirred for 2 h, and finally warmed to room temperature and stirred for 1 h. The solution was cooled to 0° C. and carefully quenched with saturated Rochelles salt solution (75 mL). The mixture was stirred vigorously for 1 h at room temperature, then extracted with ether (3×50 mL). The organic layers were combined and washed with saturated Rochelles salt solution (100 mL), H₂O (100 mL), and brine (100 mL), then dried, filtered, and concentrated. The crude material was purified by flash chromatography (20% to 40% ethyl acetate/hexanes) to give 4-(3-hydroxy-1-methyl-(Z)propenyl)-2,6-diisopropyl-pyridin-3-ol (1.81 g, 90%) as a white solid. ¹H NMR (400 MHz): δ 6.60 (s, 1H), 5.93 (dt, 1H, J=1.5, 6.4), 3.90 (d, 2H, J=7.3), 3.38 (sept., 1H, J=6.8), 2.92 (sept., 1H, J=6.8), 2.02 (d, 3H, J=1.0), 1.25 (d, 6H, J=6.8), 1.23 (d, 6H, J=6.8). ¹³C NMR (63 MHz): δ 157.6, 153.8, 143.1, 135.9, 134.8, 128.1, 116.6, 60.0, 35.3, 29.8, 24.9, 22.8(2), 21.1(2). IR (CHCl₃, cm⁻¹): 3604.3, 3528.1, 2965.5, 2870.3. MS [EI+] 246.1 (M+H)⁺, [EI−] 244.2 (M−H)⁻. Analytical (C₁₅H₂₃NO₂): Calculated C, 72.25; H, 9.30; N, 5.62. Found C, 72.30; H, 9.38; N, 5.64.

F. 3-(3-Butoxy-2,6-diisopropyl-pyridin-4-yl)-but-2(Z)-enal

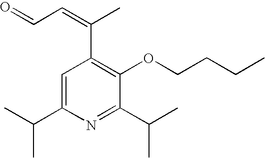

To a solution of 4-(3-hydroxy-1-methyl-(Z)-propenyl)-2,6-diisopropyl-pyridin-3-ol (271 mg, 1.09 mmol) in dimethylformamide (10 mL) were added n-iodobutane (0.14 mL, 1.23 mmol) and cesium flouride (690 mg, 4.54 mmol). The solution was stirred at room temperature for 3.5 h. Water (10 mL) was added, and the solution was stirred for an additional 30 min. then extracted with ether (3×20 mL). The organic layers were combined and washed with H₂O (20 mL) and brine (20 mL), then dried, filtered, and concentrated to give 3-(3-butoxy-2,6-diisopropyl-pyridin-4-yl)-but-2(Z)-en-1-ol (333 mg, 100%) which was used in the following reaction without flirter purification. ¹H NMR (400 MHz): δ 6.64 (s, 1H), 5.83 (dt, 1H, J=1.5), 3.79 (d, 2H, J=7.3), 3.65 (t, 2H, J=6.4), 3.38 (sept., 1H, J=6.8), 2.93 (sept. 1H, J=6.8), 2.35 (br s, 1H), 2.07 (d, 3H, J=1.5), 1.70 (m, 2H), 1.45 (m, 2H), 1.24 (d, 6H, J=6.8), 1.23 (d, 6H, J=6.8), 0.94 (t, 3H, J=7.3). MS [EI+] 306.2 (M+H)⁺.

To a 0° C. solution of 3-(3-butoxy-2,6-diisopropyl-pyridin-4-yl)-but-2(Z)-en-1-ol (300 mg, 0.98 mmol) in CH₂Cl₂ (5 mL) was added Dess-Martin periodinane (623 mg, 1.47 mmol). The solution was stirred at 0° C. for 30 min., then at room temperature for 2 h. The solution was diluted with diethyl ether (25 mL) and washed with 1N NaOH (2×20 mL) and brine (20 mL), then dried, filtered, and concentrated. The crude material was purified by flash chromatography (10% to 15% ethyl acetate/hexanes) to give 3-(3-butoxy-2,6-diisopropyl-pyridin-4-yl)-but-2(Z)-enal (270 mg, 91%) as a clear colorless oil. ¹H NMR (400 MHz): δ 9.38 (d, 1H, J=8.3), 6.70 (s, 1H), 6.10(dd, 1H, J=1.5, 8.3), 3.64 (t, 2H, J=6.4), 3.39 (sept., 1H, J=6.8), 2.97 (sept., 1H, J=6.8), 2.29 (d, 3H, J=1.0), 1.64 (m, 2H), 1.41 (m, 2H), 1.25 (d, 6H, J=6.8), 1.24 (d, 6H, J=6.8), 0.91 (t, 3H, J=7.3). ¹³C NMR (63 MHz): δ 192.9, 161.6, 160.4, 158.0, 149.7, 139.2, 129.8, 117.9, 74.4, 35.7, 32.3, 28.9, 25.5, 22.6(2), 22.1(2), 19.1, 13.8. MS [EI+] 304.2 (M+H)⁺.

G. 7-(3-Butoxy-2,6-diisopropyl-pyridin-4-yl)-3-methyl-octa-2(E),4(E),6(Z)-trienoic Acid

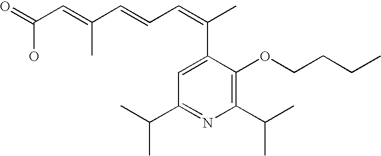

To a solution of triethyl 3-methyl-4-phosphono-crotonate (750 mg, 2.838 mmol) in tetrahydrofuran at −78° C. was added n-butyl lithium (1.85 mL, 1.6 M in hexanes). The solution was stirred at −78° C. for 20 min. to form a ylide. A solution of 3-(3-butoxy-2,6-diisopropyl-pyridin-4-yl)-but-2(Z)-enal (260 mg, 0.857 mmol) in THF (3 mL) was added to the above ylide via cannula. The resulting solution was stirred at −78 IC for 30 min., then at room temperature for 2 h under N₂ atmosphere. The solution was then diluted with H₂O (10 mL) and extracted with ethyl acetate (3×20 mL). The organic layers were combined and washed with brine (25 mL), then dried, filtered, and concentrated. The crude material was purified by flash chromatography (5% to 10% ethyl acetate/hexanes) to give 7-(3-butoxy-2,6-diisopropyl-pyridin-4-yl)-3-methyl-octa-2,4(E),6(Z)-trienoic acid ethyl ester (354 mg, 100%) as a 3:1 mixture of C2 E:Z stereoisomers, respectively. MS [EI+] 414.3 (M+H)⁺.

¹H NMR (400 MHz) data for major C2E isomer: δ 6.65 (s, 1H), 6.45 (m, 1H), 6.23 (s, 1H), 6.22 (m, 1H), 5.73 (s, 1H), 4.13 (q, 2H, J=7.3), 3.62 (t, 2H, J=6.4), 3.41 (sept., 1H, J=6.8), 2.97 (sept., 1H, J=6.8), 2.15 (s, 3H), 2.12 (d, 3H, J=1.0), 1.64 (m, 2H), 1.40 (m, 2H), 1.25 (m, 15H), 0.90 (t, 3H, J=7.3).

To a solution of the above 3:1 mixture (319 mg, 0.771 mmol) in methanol (5 mL) was added 1N NaOH (3 mL). The mixture was stirred at 45° C. for 18 h. Since TLC analysis showed a significant amount of starting material, the mixture was concentrated and dissolved in ethanol (8 mL). The solution was heated to reflux for 2 h, then cooled to room temperature, and neutralized with 1N HCl (3 mL). The mixture was extracted with ethyl acetate (3×20 mL), and the organic layers were combined and washed with brine (30 mL). The organic layer was dried, filtered, and concentrated. The crude material was purified by flash chromatography (20% to 30% ethyl acetate/hexanes) to give a 3:1 C2(E:Z) mixture of acids (276 mg, 93%) which was recrystallized from acetonitrile twice to give exclusively 7-(3-butoxy-2,6-diisopropyl-pyridin-4-yl)-3-methyl-octa-2(E),4(E),6(Z)-trienoic acid (121 mg, 41%) as a white solid. ¹H NMR (400 MHz): δ 6.64 (s, 1H), 6.49 (m, 1H), 6.24 (d, 2H, J=14.2), 5.76 (s, 1H), 3.62 (t, 2H, J=6.4), 3.41 (sept., 1H, J=6.4), 2.98 (sept., 1H, J=6.8), 2.17 (s, 3H), 2.12 (s, 3H), 1.62 (m, 2H), 1.40 (m, 2H), 1.25 (d, 12H, J=6.8), 0.90 (t, 3H, J=7.3). ¹³C NMR (63 MHz): δ 172.1, 161.1, 160.0, 155.1, 147.4, 141.2, 140.7, 134.7, 132.9, 128.4, 118.5, 118.2, 73.8, 35.7, 32.3, 29.0, 24.4, 22.7(2), 22.0(2), 19.2, 13.9, 13.8. IR (CHCl₃, cm⁻¹): 2963.2, 2934.3, 2872.4, 1679.8, 1600.3. MS [EI+] 386.3 (M+H)⁺, MS [EI−] 384.4 (M−H)⁻. Analytical (C₂₄H₃₅NO₃): Calculated C, 74.77; H, 9.15; N, 3.63. Found C, 74.91; H, 9.15; N, 3.72.

### Example 2

### 7-(2,6-Diisopropyl-3-propoxy-pyridin-4-yl)-3-methyl-octa-2(E),4(E),6(Z)-trienoic Acid

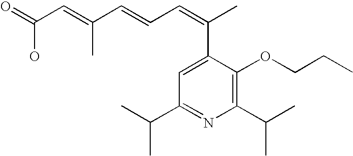

A. 3-(2,6-Diisopropyl-3-propoxy-pyridin-4-yl)-but-2(Z)-enal

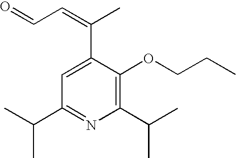

To a solution of 4-(3-hydroxy-1-methyl-(Z)propenyl)-2,6-diisopropyl-pyridin-3-ol (199 mg, 0.798 mmol) Example 1, step E) in dimethylformamide (7 mL) was added iodopropane (93 μL, 0.95 mmol) and cesium flouride (485 mg, 3.19 mmol). The mixture was stirred at room temperature for 4 h, then quenched with H₂O (5 mL). After stirring for an additional 30 min., the solution was extracted with diethyl ether (3×20 mL). The organic layers were combined, washed with H₂O (20 mL) and brine (20 mL), then dried, filtered, and concentrated. The crude 3-(2,6-diisopropyl-3-propoxy-pyridin-4-yl)-but-2(Z-en-1-ol (234 mg, 100%) was used in the next reaction without further purification. ¹H NMR (400 MHz): δ 6.64 (s, 1H), 5.82 (dt, 1H, J=1.5, 7.3), 3.78 (m, 2H), 3.62 (t, 2H, J=6.8), 3.39 (sept., 1H, J=6.8), 2.94 (sept., 1H, J=6.8), 2.34 (br s, 1H), 2.06 (d, 3H, J=1.5), 1.72 (sext., 2H, J=7.3), 1.24 (d, 6H, J=6.8), 1.23 (d, 6H, J=6.8), 0.99 (t, 3H, J=7.3). MS [EI+] 292.2 (M+H)⁺.

To a solution of 3-(2,6-diisopropyl-3-propoxy-pyridin-4-yl)-but-2(Z)-en-1-ol (239 mg, 0.798 mmol) in CH₂Cl₂ (6 mL) was added Dess-Martin periodinane (510 mg, 1.20 mmol). The solution was stirred at room temperature for 3 h, then diluted with diethyl ether (25 mL) and washed with 1N NaOH (2×10 mL) and brine (10 mL). The organic layer was dried, filtered, and concentrated. The crude material was purified by flash chromatography (5% to 10% ethyl acetate/hexanes) to give 3-(2,6-diisopropyl-3-propoxy-pyridin-4-yl)-but-2(Z)-enal (210 mg, 91%) as a clear, colorless oil. ¹H NMR (400 MHz): δ 9.38 (d, 1H, J=8.3), 6.70 (s, 1H), 6.11(dd, 1H, J 1.5, 8.3), 3.60 (t, 2H, J=6.4), 3.40 (sept., 1H, J=6.8), 2.97 (sept., 1H, J=6.8), 2.29 (d, 3H, J=1.5), 1.68 (sext., 2H, J=7.3), 1.25 (d, 6H, J=6.8), 1.24 (d, 6H, J=6.8), 0.96 (t, 3H, J=7.3). MS [EI+] 290.2 (M+H)⁺.

B. 7-(2,6-Diisopropyl-3-propoxy-pyridin-4-yl)-3-methyl-octa-2(E),4(E),6(Z)-trienoic acid

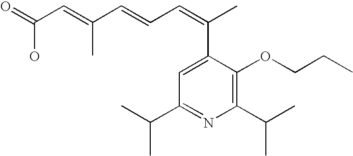

To a solution of triethyl 3-methyl-4-phosphono-crotonate (617 mg, 2.838 mmol) in tetrahydrofuran (5 mL) at −78° C. was added n-butyl lithium (1.50 mL, 1.6 M in hexanes). The solution was stirred at −78° C. for 20 min. to form a ylide. A solution of 3-(2,6-diisopropyl-3-propoxy-pyridin-4-yl)-but-2(Z)-enal (207 mg, 0.715 mmol) in tetrahydrofuran (3 mL) was added to the above ylide via cannula. The resulting solution was stirred at −78° C. for 30 min. then at room temperature for 2 h under N₂ atmosphere. The solution was then diluted with H₂O (10 mL) and extracted with ethyl acetate (3×20 mL). The organic layers were combined and washed with water (20 mL) and brine (20 mL), then dried, filtered, and concentrated. The crude material was purified by flash chromatography (0 to 8% ethyl acetate/hexanes) to give 7-(2,6-diisopropyl-3-propoxy-pyridin-4-yl)-3-methyl-octa-2,4(E),6(Z)-trienoic acid ethyl ester (278 mg, 97%) as a 3:1 mixture of C2 E:Z stereoisomers, respectively. MS [EI+] 400.3 (M+H)⁺.

¹H NMR (400 MHz) data for major C2E isomer: δ 6.65 (s, 1H), 6.45 (m, 1H), 6.24 (s, 1H), 6.22 (m, 1H), 5.73 (s, 1H), 4.13 (q, 2H, J=7.3), 3.58 (t, 2H, J=6.2), 3.42 (sept., 1H, J=6.6), 2.97 (sept., 1H, J=7.0), 2.16 (s, 3H), 2.12 (d, 3H, J=1.2), 1.66 (sext., 2H, J=7.4), 1.24 (m, 15H), 0.91 (t, 3H, J=7.4).

To a solution of the above 3:1 mixture (272 mg, 0.681 mmol) in ethanol (5.5 mL) was added 1N NaOH (2.6 mL). The mixture was heated to 90° C. and stirred for 3 h, then cooled to room temperature, and neutralized with 1N HCl (2.6 mL). The mixture was extracted with ethyl acetate (3×20 mL), and the organic layers were combined and washed with brine (20 mL). The organic layer was dried, filtered, and concentrated. The crude material was purified by flash chromatography (20% to 30% ethyl acetate/hexanes) to give a mixture of C2 EZ acids (219 mg, 86%) which was recrystallized from acetonitrile twice to give exclusively 7-(2,6-diisopropyl-3-propoxy-pyridin-4-yl)-3-methyl-octa-2(E),4(E),6(Z)-trienoic acid (96 mg, 38%) as a white solid. ¹H NMR (400 MHz): δ 6.66 (s, 1H), 6.50 (m, 1), 6.25 (d, 1H, J=15.2), 6.25 (m, 1H), 5.76 (s, 1H), 3.58 (t, 2H, J=6.2), 3.43 (sept., 1H, J=6.6), 2.99 (sept., 1H, J=6.6), 2.17 (s, 3H), 2.13 (s, 3H), 1.66 (m, 2H), 1.25 (d, 6H, J=6.6), 1.24 (d, 6H, J=6.6), 0.95 (t, 3H, J=7.4). ¹³C NMR (63 MHz): δ 172.2, 161.1, 160.0, 155.0, 147.4, 141.2, 140.6, 134.7, 132.9, 128.4, 118.5, 118.2, 75.6, 35.7, 29.0, 24.4, 23.5, 22.7(2), 22.0(2), 13.9, 10.6. IR (CHCl₃, cm⁻¹): 2965.5, 2937.8, 1680.5, 1600.5. MS [EI+] 372.2 M+H)⁺, MS [EI−] 370.3 (M−H)⁻. Analytical (C₂₃H₃₃NO₃): Calculated C, 74.36; H, 8.95; N, 3.77. Found C, 74.04; H, 8.66; N, 3.93.

### Example 3

### 7-(2,6-Diisopropyl-3-ethoxy-pyridin-4-yl)-3-methyl-octa-2(E),4(E),6(Z)-trienoic acid

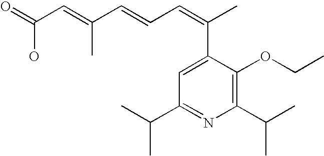

A. 3-(2,6-Diisopropyl-3-ethoxy-pyridin-4-yl)-but-2(Z)-enal

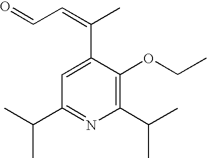

To a solution of 4-(3-hydroxy-1-methyl-(Z)propenyl)-2,6-diisopropyl-pyridin-3-ol (199 mg, 0.798 mmol) (see Example 1, step E) in dimethylformamide (7 mL) was added iodoethane (77 μL, 0.96 mmol) and cesium flouride (490 mg, 3.22 mmol). The mixture was stirred at room temperature for 4 h, then quenched with H₂O (5 mL). After stirring for an additional 30 min., the solution was extracted with diethyl ether (3×20 mL). The organic layers were combined, washed with H₂O (20 mL) and brine (20 mL), then dried, filtered, and concentrated. The crude 3-(2,6-diisopropyl-3-ethoxy-pyridin-4-yl)-but-2(Z)-en-1-ol (224 mg, 100%) was used in the next reaction without further purification. ¹H NMR (400 MHz): δ 6.64 (s, 1), 5.83 (dt, 1H, J=1.5, 7.3), 3.79 (t, 2H, J=6.8), 3.73 (q, 2H, J=6.8), 3.39 (sept., 1H, J=6.8), 2.94 (sept., 1H, J=6.8), 2.41 (br t, 1H, J=5.9), 2.07 (d, 3H, J=1.0), 1.33 (t, 3H, J=7.3), 1.24 (d, 6H, J=6.8), 1.23 (d, 3H, J=6.8). MS [EI+] 278.2 (M+H)⁺.

To a solution of 3-(2,6-diisopropyl-3-ethoxy-pyridin-4-yl)-but-2(Z)-en-1-ol (224 mg, 0.798 mmol) in CH₂Cl₂ (6 mL) was added Dess-Martin periodinane (524 mg, 1.24 mmol). The solution was stirred at room temperature for 3 h, then diluted with diethyl ether (25 mL) and washed with 1N NaOH (2×10 mL) and brine (10 mL). The organic layer was dried, filtered, and concentrated. The crude material was purified by flash chromatography (5% to 10% ethyl acetate/hexanes) to give 3-(2,6-diisopropyl-3-propoxy-pyridin-4-yl)-but-2(Z)-enal (199 mg, 91%) as a clear, colorless oil. ¹H NMR (400 MHz): δ 9.39 (d, 1H, J=7.8), 6.70 (s, 1H), 6.12(d, 1H, J=1.5, 8.3), 3.72 (q, 2H, J=6.8), 3.40 (sept., 1H, J=6.8), 2.97 (sept., 1H, J=7.3), 2.29 (d, 3H, J=1.5), 1.29 (t, 311, J=6.8), 1.25 (d, 6H, J=6.8), 1.24 (d, 6H, J=6.8). MS [EI+] 276.1 (M+H)⁺.

B. 7-(2,6-Diisopropyl-3-ethoxy-pyridin-4-yl)-3-methyl-octa-2(E),4(E),6(Z)-trienoic acid

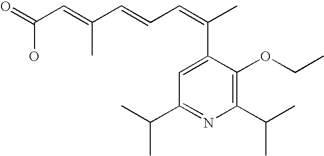

To a solution of triethyl 3-methyl-4-phosphono-crotonate (627 mg, 2.37 mmol) in tetrahydrofuran (5 mL) at −78° C. was added n-butyl lithium (1.50 mL, 1.6 M in hexanes). The solution was stirred at −78° C. for 20 min. to form a ylide. A solution of 3-(2,6-diisopropyl-3-ethoxy-pyridin-4-yl)-but-2(Z)-enal (199 mg, 0.723 mmol) in tetrahydrofuran (3 mL) was added to the above ylide via cannula. The resulting solution was stirred at −78° C. for 30 min., then at room temperature for 2.5 h under N₂ atmosphere. The solution was then diluted with H₂O (10 mL) and extracted with ethyl acetate (3×20 mL). The organic layers were combined, washed with water (20 mL) and brine (20 mL), then dried, filtered, and concentrated. The crude material was purified by flash chromatography (0 to 8% ethyl acetate/hexanes) to give 7-(2,6-diisopropyl-3-ethoxy-pyridin-4-yl)-3-methyl-octa-2,4(E),6(Z)-trienoic acid ethyl ester (256 mg, 92%) as a 3:1 mixture of C2 E:Z stereoisomers, respectively. MS [EI+] 386.3 (M+H)⁺.

¹H NMR (400 MHz) data for major C2E isomer: δ 6.65 (s, 1H), 6.46 (m, 1H), 6.24 (s, 1H), 6.22 (m, 11), 5.73 (s, 1H), 4.13 (q, 2H, J=7.0), 3.69 (q, 2H, J=7.0), 3.43 (sept., 11, J=7.0), 2.97 (sept., 1H, J=7.0), 2.16 (s, 3H), 2.12 (d, 3H, J=1.2), 1.26 (m, 18H).

To a solution of the above 3:1 mixture (249 mg, 0.646 mmol) im ethanol (5.5 mL) was added 1N NaOH (2.5 mL). The mixture was heated to 90° C. and stirred for 3 h then cooled to room temperature and neutralized with 1N HCl (2.5 mL). The mixture was extracted with ethyl acetate (3×20 mL), and the organic layers were combined and washed with brine (20 mL). The organic layer was dried, filtered, and concentrated. The crude material was purified by flash chromatography (20% to 30% ethyl acetate/hexanes) to give a mixture of C2 E,Z acids (207 mg, 90%) which was recrystallized from acetonitrile twice to give exclusively 7-(2,6-diisopropyl-3-ethoxy-pyridin-4-yl)-3-methyl-octa-2(E),4(E),6(Z)-trienoic acid (120 mg, 52%) as a white solid. ¹H NMR (400 MHz): δ 6.66 (s, 1H), 6.50 (m, 1H), 6.25 (m, 2H), 5.76 (s, 1H), 3.70 (m, 2H), 3.44 (sept., 1H, J=7.0), 2.99 (sept., 1H, J=7.0), 2.17 (s, 3H), 2.13 (s, 3H), 1.26 (m, 15H). ¹³C NMR (63 MHz): δ 172.3, 161.2, 160.1, 155.0, 147.4, 141.3, 140.8, 134.8, 132.8, 128.5, 118.6, 118.4, 69.6, 35.7, 29.0, 24.2, 22.7(2), 22.0(2), 15.8, 13.9. IR (CHCl₃, cm⁻¹): 2965.9, 2929.3, 2870.7, 1680.3, 1600.3. MS [EI+] 358.2 (M+H)⁺, MS [EI−] 356.3 (M−H)⁻. Analytical (C₂₂H₃₁NO₃): Calculated C, 73.92; H, 8.74; N, 3.92. Found C, 74.02; H, 8.60; N, 4.03.

### Example 4

### 7-[3-(2,2-Difluoro-ethoxy)-2,6-diisopropyl-pyridin-4-yl]-3-methyl-octa-2(E),4(E),6(Z)-trienoic acid

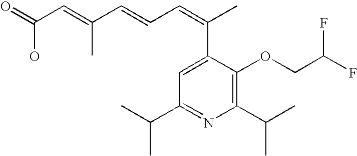

A. 3-[3-(2,2-Difluoro-ethoxy)-2,6-diisopropyl-pyridin-4-yl]-but-2(Z)-enal

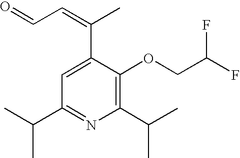

To a solution of 4-(3-hydroxy-1-methyl-(Z)propenyl)-2,6-diisopropyl-pyridin-3-ol (201 mg, 0.806 mmol) (see Example 1, step E) in dimethylformamide (7 mL) was added 2-bromo-1,1-difluoro-ethane (0.150 mL, 1.89 mmol) and cesium flouride (520 mg, 3.42 mmol). The mixture was stirred at room temperature for 18 h, then quenched with H₂O (5 mL). After stirring for an additional 30 min., the solution was extracted with diethyl ether (3×20 mL). The organic layers were combined, washed with H₂O (20 mL) and brine (20 mL), then dried, filtered, and concentrated. The crude 3-[3-(2,2-difluoro-ethoxy)-2,6-diisopropyl-pyridin-4-yl]-but-2(Z)-en-1-ol was used in the next reaction without further purification. ¹H NMR (400 MHz): δ 6.66 (s, 1H), 5.98 (tt, 1H, J=3.9, 55.2) 5.82 (dt, 1H, J=1.5, 7.3), 3.88 (m, 4H), 3.38 (sept., 1H, J=6.8), 2.94 (sept., 1H, J=6.8), 2.07 (d, 3H, J=1.5), 1.67 (br s, 1H), 1.24 (d, 6H, J=6.8), 1.23 (d, 6H, J=6.8). MS [EI+] 314.1 (M+H)⁺.

To a solution of 3-[3-(2,2-difluoro-ethoxy)-2,6-diisopropyl-pyridin-4-yl]-but-2(Z)-en-1-ol in CH₂Cl₂ (5 mL) was added Dess-Martin periodinane (519 mg, 1.22 mmol). The solution was stirred at room temperature for 3 h, then diluted with diethyl ether (25 mL) and washed with 1N NaOH (2×10 mL) and brine (10 mL). The organic layer was dried, filtered, and concentrated. The crude material was purified by flash chromatography (5% to 10% ethyl acetate/hexanes) to give 3-[3-(2,2-difluoro-ethoxy)-2,6-diisopropyl-pyridin-4-yl]-but-2(Z)-enal (213 mg, 85%) as a clear, colorless oil. ¹H NMR (400 MHz): δ 9.38 (d, 1H, J=8.3), 6.74 (s, 1H), 6.16(m, 1H), 5.94 (tt, 1H, J=3.9, 54.7), 3.88 (dt, 2H, J=3.9, 13.4), 3.38 (sept., 1H, J=6.8), 2.99 (sept., 1H, J=6.8), 2.30 (d, 3H, J=1.5), 1.25 (d, 6H, J=6.8), 1.25 (d, 6H, J=6.8). MS [EI+] 312.1 (M+H)⁺, [EI−] 310.2 (M−H)⁻.

B. 7-[3-(2,2-Difluoro-ethoxy)-2,6-diisopropyl-pyridin-4-yl]-3-methyl-octa-2(E),4(E),6(Z)-trienoic acid

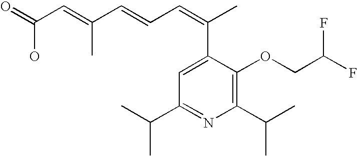

To a solution of triethyl 3-methyl-4-phosphono-crotonate (592 mg, 2.24 mmol) in tetrahydrofuran at −78° C. (4 mL) was added n-butyl lithium (1.40 mL, 1.6 M in hexanes). The solution was stirred at −78° C. for 20 min. to form a ylide. A solution of 3-[3-(2,2-difluoro-ethoxy)-2,6-diisopropyl-pyridin-4-yl]-but-2(Z)-enal (211 mg, 0.678 mmol) in tetrahydrofuran (3 mL) was added to the above ylide via cannula. The resulting solution was stirred at −78° C. for 30 min., then at room temperature for 3 h under N₂ atmosphere. The solution was then diluted with H₂O (10 mL) and extracted with ethyl acetate (3×20 mL). The organic layers were combined, washed with water (20 mL) and brine (20 mL), then dried, filtered, and concentrated. The crude material was purified by flash chromatography (0 to 8% ethyl acetate/hexanes) to give 7-[3-(2,2-difluoro-ethoxy)-2,6-diisopropyl-pyridin-4-yl]-3-methyl-octa-2,4,6-trienoic acid ethyl ester (239 mg, 84%) as a 3:1 mixture of C2 E:Z stereoisomers, respectively. MS [EI+] 422.2 (M+H)⁺.

¹H NMR (400 MHz) data for major C2E isomer: δ 6.68 (s, 1H), 6.40 (m, 1H), 6.27 (m, 2H), 5.92 (tt, 1H, J=4.3, 51.2), 5.75 (s, 1H), 4.13 (q, 2H, J=7.0), 3.85 (dt, 2H, J=3.9, 13.7), 3.41 (sept., 1H, J=6.6), 2.98 (sept., 1H, J=6.6), 2.16 (s, 3H), 2.11 (d, 3H, J=1.2), 1.25 (m, 9H), 1.25 (d, 6H, J=7.0).

To a solution of the above 3:1 mixture (232 mg, 0.550 mmol) in ethanol (5 mL) was added 1N NaOH (2.2 mL). The mixture was heated to 90° C. and stirred for 3 h, then cooled to room temperature and neutralized with 1N HCl (2.2 mL). The mixture was extracted with ethyl acetate (3×20 mL), and the organic layers were combined and washed with brine (20 mL). The organic layer was dried, filtered, and concentrated. The crude material was purified by flash chromatography (20% to 30% ethyl acetate/hexanes) to give a mixture of C2 EZ acids (190 mg, 88%) which was recrystallized from acetonitrile twice to give exclusively 7-[3-(2,2-difluoro-ethoxy)-2,6-diisopropyl-pyridin-4-yl]-3-methyl-octa-2(E),4(E),6(Z)-trienoic acid (34 mg, 16%) as a white solid. ¹H NMR (400 MHz): 36.69 (s, 1H), 6.45 (m, 1H), 6.29 (m, 2H), 5.93 (tt, 1H, J=3.9, 55.1), 5.77 (s, 1H), 3.85 (dt, 2H, J=4.3, 13.7), 3.41 (sept., 1H, J=7.0), 3.00 (sept., 1H, J=7.0), 2.17 (s, 3E), 2.12 (s, 3H), 1.26 (d, 6H, J=6.6), 1.25 (d, 6H, J=7.0). ¹³C NMR (63 MHz): δ 172.0, 162.4, 159.6, 154.7, 146.2, 140.8, 139.2, 135.6, 132.0, 129.2, 118.8, 118.7, 113.6 (t, 1C, J=241), 72.0 (t, 1C, J=28), 35.8, 29.0, 24.2, 22.6(2), 21.9(2), 13.9. IR (CHCl₃, cm⁻): 2966.4, 2930.3, 2871.5, 1682.5, 1602.8. MS [EI+] 394.2 (M+H)⁺, MS [EI−] 392.3 (M−H)⁻. Analytical (C₂₂H₂₉F₂NO₃): Calculated C, 67.16; H, 7.43; N, 3.56. Found C, 67.31; H, 7.47; N, 3.51.

### Example 5

### 7-[2,6-Diisopropyl-3-(2,2,2-trifluoro-ethoxy)-pyridin-4-yl]-3-methyl-octa-2(E),4(E),6(Z)-trienoic acid

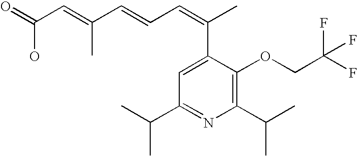

A. 3-[2,6-Diisopropyl-3-(2,2,2-trifluoro-ethoxy)-pyridin-4-yl]-but-2(Z)-enal

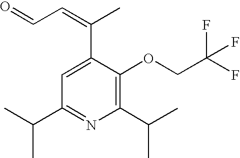

To a solution of 4-(3-hydroxy-1-methyl-(Z)propenyl)-2,6-diisopropyl-pyridin-3-ol (209 mg, 0.838 mmol) (see Example 1, step E) in dimethylformamide (7 mL) was added 2-bromo-1,1,1-trifluoro-ethane (0.10 mL, 1.1 mmol) and cesium flouride (540 mg, 3.55 mmol). The mixture was stirred at room temperature for 2 h. The mixture was then transferred to a sealed tube containing dimethylformamide (1 mL) and cesium carbonate (547 mg, 1.68 mmol) and heated to 50° C. overnight. The reaction was quenched with H₂O (10 mL). After stirring for an additional 30 mm, the solution was extracted with diethyl ether (3×20 mL). The organic layers were combined, washed with H₂O (20 mL) and brine (20 mL), then dried, filtered, and concentrated. The crude material was purified by flash chromatography (5% to 20% ethyl acetate/hexanes) to give 3-[2,6-diisopropyl-3-(2,2,2-trifluoro-ethoxy)-pyridin-4-yl]-but-2(Z)-en-1-ol (60 mg, 22%) as a light yellow oil. ¹H NMR (400 MHz): δ 6.66 (s, 1H), 5.82 (dt, 1H, J=1.6, 7.4), 4.06 (q, 2H, J=8.6), 3.90 (d, 2H, J=7.0), 3.40 (sept., 1H, J=6.6), 2.96 (sept., 1H, J=7.0), 2.07 (d, 3H, J=1.2), 1.64 (br s, 1H), 1.24 (d, 6H, J=7.0), 1.23 (d, 6H, J=6.6). MS [EI+] 332.1 (M+H)⁺.

To a solution of 3-[2,6-diisopropyl-3-(2,2,2-trifluoro-ethoxy)-pyridin-4-yl]-but-2(z)-en-1-ol (60 mg, 0.181 mmol) in CH₂Cl₂ (2 mL) was added Dess-Martin periodinane (129 mg, 0.304 mmol). The solution was stirred at room temperature for 1.5 h, then diluted with diethyl ether (20 mL) and washed with 1N NaOH (2×5 mL) and brine (10 mL). The organic layer was dried, filtered, and concentrated. The crude material was purified by flash chromatography (0 to 10% ethyl acetate/hexanes) to give 3-[2,6-diisopropyl-3-(2,2,2-trifluoro-ethoxy)-pyridin-4-yl]-but-2(Z)-enal (44 mg, 74%) as a clear, colorless oil. ¹H NMR (400 MHz): δ 9.40 (d, 1H, J=8.2), 6.74 (s, 1H), 6.17(m, 1H), 4.03 (m, 2H), 3.38 (sept., 1H, J=7.0), 2.99 (sept., 1H, J=7.0), 2.30 (d, 3H, J=1.2), 1.25 (d, 12H, J=7.0). MS [EI+] 330.1 (M+H)⁺, [EI−] 328.1 (M−H)⁻.

B. 7-[2,6-Diisopropyl-3-(2,2,2-trifluoro-ethoxy)-pyridin-4-yl]-3-methyl-octa-2(E),4(E),6(Z)-trienoic acid

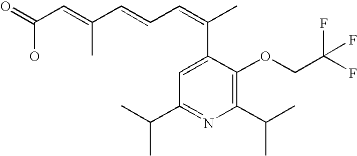

To a −78° C. solution of triethyl 3-methyl-4-phosphono-crotonate (129 mg, 0.488 mmol) in THF (1 mL) was added n-butyl lithium (0.29 mL, 1.6 M in hexanes). The solution was stirred at −78° C. for 20 min. to form a ylide. A solution of 3-[2,6-diisopropyl-3-(2,2,2-trifluoro-ethoxy)-pyridin-4-yl]-but-2(Z)-enal (44 mg, 0.13 mmol) in THF (0.5 mL+2×0.5 mL rinse) was added to the above ylide via cannula. The resulting solution was stirred at −78° C. for 30 min., then at room temperature for 3 h under N₂ atmosphere. The solution was then diluted with H₂O (10 mL) and extracted with ethyl acetate (3×20 mL). The organic layers were combined, washed with water (20 mL) and brine (20 mL), then dried, filtered, and concentrated. The crude material was purified by flash chromatography (0 to 8% ethyl acetate/hexanes) to give 7-[2,6-diisopropyl-3-(2,2,2-trifluoro-ethoxy)-pyridin-4-yl]-3-methyl-octa-2,4,6-trienoic acid ethyl ester (53 mg, 90%) as a 3:1 mixture of C2 E:Z stereoisomers, respectively. MS [EI+] 440.2 (M+H)⁺.

¹H NMR (400 MHz) data for major C2 E isomer: δ 6.69 (s, 1H), 6.36 (m, 1H), 6.28 (m, 1H), 5.76 (s, 1H), 4.14 (q, 2H, J=7.0), 3.99 (q, 2H, J=8.2), 3.43 (sept., 1H, J=7.0), 2.99 (sept., 1H, J=7.0), 2.16 (s, 3H), 2.11 (d, 3H, J=0.8), 1.25 (m, 9H), 1.24 (d, 6H, J=6.6).

To a solution of the above 3:1 mixture (53 mg, 0.121 mmol) in ethanol (1.0 mL) was added 1N NaOH (0.36 mL). The mixture was heated to 90° C. and stirred for 3 h then cooled to room temperature, diluted with H₂O (10 mL), and neutralized with 1N HCl (0.36 nm). The mixture was extracted with ethyl acetate (3×15 mL) and the organic layers were combined and washed with H₂O (10 mL) and brine (10 mL). The organic layer was dried, filtered, and concentrated. The crude material was purified by flash chromatography (20% to 30% ethyl acetate/hexanes) to give a mixture of C2 E,Z acids (49 mg, 100%) which was recrystallized from acetonitrile twice to give exclusively 7-[3-(2,2-difluoro-ethoxy)-2,6-diisopropyl-pyridin-4-yl]-3-methyl-octa-2(E),4(E),6(Z)-trienoic acid (12 mg, 24%) as a white solid. ¹H NMR (400 MHz): δ 6.69 (s, 1H), 6.44 (dd, 1H, J=10.6, 14.8), 6.30 (m, 2H), 5.78 (s, 1H), 3.99 (q, 2H, J=8.6), 3.43 (sept., 1H, J=6.6), 3.00 (sept., 1H, J=7.0), 2.17 (s, 3H), 2.12 (d, 3H, J=0.8), 1.25 (d, 12H, J=6.6). IR (CHCl₃, cm⁴¹): . MS [EI+] 412.2 (M+H)⁺, MS [EI−] 410.1 (M−H)⁻. Analytical (C₂₂H₂₈F₃NO₃): Calculated C, 64.22; H, 6.86; N, 3.40. Found C,; H,; N,.

Biological Activity

### Example 6

### Evaluation of Retinoid Receptor Subfamily Activity In Vitro

Utilizing the “cis-trans” or “co-transfection” assay described by Evans et al., Science, 240:889-95 (ay 13, 1988), the disclosure of which is herein incorporated by reference, the dimer-selective RXR modulator compounds of the present invention were tested and found to have strong, specific activity as selective RXR modulators, including activity as full agonists, partial agonists and/or full antagonists of RXR homodimers and/or heterodimers. This assay is described in further detail in U.S. Pat. Nos. 4,981,784 and 5,071,773, the disclosures of which are incorporated herein by reference.

The co-transfection assay provides a method for identifying functional agonists which mimic, or antagonists which inhibit, the effect of native hormones, and quantifying their activity for responsive IR proteins. In this regard, the co-transfection assay mimics an in vivo system in the laboratory. Importantly, activity in the co-transfection assay correlates very well with known in vivo activity, such that the co-transfection assay functions as a qualitative and quantitative predictor of a tested compounds in vivo pharmacology. See, e.g., T. Berger et al. 41 J. Steroid Biochem. Molec. Biol. 773 (1992), the disclosure of which is herein incorporated by reference.

In the co-transfection assay, cloned cDNA for one or more IRs (e.g., human RARα, RXRα, or PPARγ), alone or in combination (i.e. for heterodimer assays) under the control of a constitutive promoter (e.g., the SV 40, RSV or CMV promoter) is introduced by transfection (a procedure to introduce exogenous genes into cells) into a background cell substantially devoid of endogenous IRs. These introduced gene(s) direct the recipient cells to make the IR protein(s) of interest. A further gene is also introduced (co-transfected) into the same cells in conjunction with the IR gene(s). This further gene, comprising the cDNA for a reporter protein, such as firefly luciferase (LUC), controlled by an appropriate hormone responsive promoter containing a hormone response element (HRE). This reporter plasmid functions as a reporter for the transcriptional-modulating activity of the target IR(s). Thus, the reporter acts as a surrogate for the products (mRNA then protein) normally expressed by a gene under control of the target receptor(s) and their native hormone(s).

The co-transfection assay can detect small molecule agonists or antagonists, including partial agonists and antagonist, of target IRs. Exposing the transfected cells to an agonist ligand compound increases reporter activity in the transfected cells. This activity can be conveniently measured, e.g., by increasing luciferase production and enzymatic activity, which reflects compound-dependent, IR-mediated increases in reporter transcription. To detect antagonists, the co-transfection assay is carried out in the presence of a constant concentration of an known agonist to the target IR (e.g., 4-[(3,5,5,8,8-Pentamethyl-5,6,7,8-tetrahydro-2-naphthyl)ethenyl]benzoic acid (Ligand Pharmaceuticals, Inc.) for RXRα) known to induce a defined reporter signal. Increasing concentrations of an antagonist will decrease the reporter signal (e.g., luciferase production). The co-transfection assay is therefore useful to detect both agonists and antagonists of specific IRs. Furthermore, it determines not only whether a compound interacts with a particular IR, but whether this interaction mimics (agonizes) or blocks (antagonizes) the effects of native or synthetic regulatory molecules on target gene expression, as well as the specificity and strength of this interaction.

The activity of the dimer-selective RXR retinoid modulator compounds of the present invention were evaluated utilizing the co-transfection assay according to the following illustrative Examples.

### Example 6A

### RXR Homodimer Co-Transfection Assay

CV-1 cells (African green monkey kidney fibroblasts) were cultured in the presence of Dulbecco's Modified Eagle Medium (DMEM) supplemented with 10% charcoal resin-stripped fetal bovine serum then transferred to 96-well microtiter plates one day prior to transfection.

To determine agonist and antagonist activity of the modulator compounds of the present invention, the CV-1 cells were transiently transfected by calcium phosphate coprecipitation according to the procedure of Berger et al., J. Steroid Biochem. Mol. Biol. 41:733 (1992) with the receptor expressing plasmid pRShRXRα, Mangelsdorf et al., Nature, 345:224 (1990), the disclosures of which are herein incorporated by reference, at a concentration of 10 ng/well. The receptor expression plsmid was cotransfected along with a reporter plasmid at 50 ng/well, the internal control plasmid pRS-β-Gal at 50 ng/well and filler DNA, pGEM, at 90 ng/well.

The reporter plasmid CRBPIITKLUC, which contains an RXRE (retinoid X receptor response element, as described in Mangelsdorf et al., Cell, 66:555 (1991), the disclosure of which is herein incorporated by reference, was used in transfections for the RXR homodimer assay. This reporter plasmid contains the cDNA for firefly luciferase (LUC) under the control of a promoter containing the RXR response element. As noted above, pRS-β-Gal, coding for constitutive expression of E. coli β-galactosidase (β-Gal), was included as an internal control for evaluation of transfection efficiency and compound toxicity.

Six hours after transfection, media was removed and the cells were washed with phosphate-buffered saline (PBS). Media containing compounds of the present invention in concentrations ranging from 10⁻¹⁰ to 10⁻⁵ M were added to the cells. Similarly, the reference compounds all-trans retinoic acid (ATRA)(Sigma Chemical), (4-[3,5,5,8,8-Pentamethyl-5,6,7,8-tetrahydro-2-naphthyl)ethenyl]benzoic acid: Ligand Pharmaceuticals, Inc.) and (6-[1-(3,5,5,8,8-pentamethyl-5,6,7,8-tetrahydronaphthalen-2-yl)cyclopropyl]nicotinic acid: Ligand Pharmaceuticals, Inc.), compounds with known agonist activity on RXRs, were added at similar concentrations to provide a reference point for analysis of the agonist activity of the compounds of the present invention. When determining the antagonist activity of the compounds of the present invention, the compounds were added to the cells in the presence of a fixed concentration (3.2×10⁻⁸ M) of the known RXR agonist (4-[3,5,5,8,8-Pentamethyl-5,6,7,8-tetrahydro-2-naphthyl)ethenyl]benzoic acid: Ligand Pharmaceuticals, Inc.). Retinoid purity was established as greater than 99% by reverse phase high-performance liquid chromatography. Retinoids were dissolved in dimethylsulfoxide for use in the transcriptional activation assays. Two to three replicates were used for each sample. Transfections and subsequent procedures were performed on a Biomek 1000 automated workstation.

After 40 hours, the cells were washed with PBS, lysed with a detergent-based buffer and assayed for LUC and β-Gal activities using a luminometer or spectrophotometer, respectively. For each replicate, the normalized response (NR) was calculated as:

LUC response/β-Gal rate

where β-Gal rate=β-Gal·1×10⁵/β-Gal incubation time.

The mean and standard error of the mean (SEM) of the NR were calculated. Data were plotted as the response of the compound compared to the reference compounds over the range of the dose-response curve. For the agonist activity of the compounds of the present invention, the effective concentration that produced 50% of the maximum response (EC₅₀) was quantified. Antagonist activity was determined by testing the amount of LUC expression in the presence of the RXR agonists described above at the EC₅₀ concentration for such known compounds. The concentration of compounds of the present invention that inhibited 50% of LUC expression induced by the reference agonist was quantified (IC₅₀). In addition, the efficacy of antagonists was determined as a function (%) of maximal inhibition.

The RXRα binding activity and agonist and antagonist activity in the RXRα homodimer cotransfection assay of selected compounds of the present invention are shown in Table 1 below.

TABLE 1
Activity of RXR modulators of present invention in
the RXR□ homodimer cotransfection assays.
EC50 and IC50 values were not calculated if
efficacy was <10%. Values represent the mean
of n > 2 independent experiments.

RXRα Homodimer Cotransfection Assay

| | Agonist | Agonist | Antagonist | |
|---|---|---|---|---|
| | Efficacy | EC₅₀ | Efficacy | Antagonist |
| Example | (%) | (nM) | (%) | IC₅₀ (nM) |
| 3 | 51 | 2 | 3 | — |
| 2 | 4 | — | 88 | 4.1 |
| 1 | 1 | — | 93 | 3.5 |
| 4 | 9 | — | 76 | 8.7 |
| 5 | 3 | — | 85 | 8 |

As can be seen in Table 1, Compound 3 displayed agonist activity; and Compounds 1-2 and 4-5 displayed highly efficacious and potent antagonist activity with little or no agonists activity. Thus, compounds of the present invention display properties ranging from full agonists to full antagonists in the context of RXR homodimers.

### Example 6B

### RXR and RAR Binding

In addition to the cotransfection data, the binding of selected compounds of the present invention to the RAR and RXR receptors was also investigated according to the methodology described in M. F. Boehm, et al, “Synthesis and Structure-Activity Relation-ships of Novel Retinoid X Receptor Selective Retinoids,” J. Med. Chem., 37:2930 (1994); M. F. Boehm, et al., “Synthesis of High Specific Activity [³H]-9-cis Retinoic Acid and Its Application for Identifying Retinoids with Unusual Binding Properties,” J. Med. Chem., 37:408 (1994), and E. A. Allegretto, et al., “Characterization and Comparison of Hormone-Binding and Transactivation Properties of Retinoic Acid and Retinoid X Receptors Expressed in Mammalian Cells and Yeast,” J. Biol. Chem., 268:22625 (1993), the disclosures of which are herein incorporated by reference.

Non-specific binding was defined as that binding remaining in the presence of 500 nM of the appropriate unlabelled compound. At the end of the incubation period, bound ligand was separated from free. The amount of bound tritiated retinoid was determined by liquid scintillation counting of an aliquot (700 μL) of the supernatant fluid or the hydroxylapatite pellet.

After correcting for non-specific binding, IC₅₀ values were determined. The IC₅₀ value is defined as the concentration of competing ligand needed to reduce specific binding by 50%. The IC₅₀ value was determined graphically from a log-logit plot of the data. The Kd values were determined by the application of the Cheng-Prussof equation to the IC₅₀ values, the labeled ligand concentration and the K_{d} of the labeled liand.

### Example 6C

### RXR Heterodimer Co-Transfection Assays

The compounds of the present invention were further tested for activity on RXR heterodimers with RARα, RARγ or PPARγ utilizing the cotransfection assay in CV-1 cells as described in Example 6A. The RXR:RAR heterodimer cotransfection assays utilized the following expression plasmids and reporter plasmid: pRShRARα (10 ng/well, Giguere et al., Nature, 330:624 (1987) the disclosure of which is herein incorporated by reference) or pRShRARγ (10 ng/well, Ishikawa et al., Mol. Endocrin., 4:837 (1990) the disclosure of which is herein incorporated by reference) with Δ-MTV-LUC (50 ng/well, Hollenberg and Evans, Cell, 55:899 (1988), the disclosure of which is herein incorporated by reference) containing an RARE which is referred to as two copies of the TRE-palindromic response element described in Umesono et al., Nature, 336:262 (1988), the disclosure of which is herein incorporated by reference. For the RSR:PPARγ heterodimer cotransfection assay, the RXRα receptor expression plasmid, pRShRXRα (10 ng/well), was cotrasfected with the PPARγ expression plasmid, pCMVhPPARγ (10 ng/well), and a reporter plasmid containing three copies of a PPARγ response element (pPREA3-tk-LUC, 50 ng/well; Mukherjee et al., Journ. Biol. Chem., 272:8071-8076 (1997) and references cited therein, the disclosures of which are herein incorporated by reference).

Cotransfections were performed as described in Example 6A. For determination of agonist activity in the context of the RXR:RAR heterodimer or the RXR:PPARγ heterodimer, media containing compounds of the present invention in concentrations ranging from 10⁻¹⁰ to 10⁻⁵ M were added to the cells. Similarly, the reference compounds all-trans retinoic acid (ATRA)(Sigma Chemical) and TTNPB ((E)-4-[2-(5,6,7,8-tetrahydro-5,5,8,8-tetramethyl-2-naphthalenyl)-1-propenyl]benzoic acid: Hoffman LaRoche, Inc.), known RAR agonist compounds, or BRL 49653, a compound with known agonist activity on PPARγ, were added at similar concentrations to provide a reference point for analysis of the agonist activity of the compounds of the present invention. When evaluating the antagonist activity of the compounds of the present invention on RARγ, the compounds were added to the cells in the presence of a fixed concentration (1×10⁻⁸ M) of the known RAR selective agonist TTNPB ((E)-4[2-(5,6,7,8-tetrahydro-5,5,8,8-tetramethyl-2-naphthalenyl)-1-propenyl]benzoic acid: Hoffman LaRoche, Inc.). Antagonist efficacy and IC₅₀ values were determined as in Example 6A.

Compounds of the present invention were also tested for the ability to synergize with an RAR or PPARγ agonist in the context of an RXR heterodimer. For these assays the compounds were added to the cells with a fixed concentration of TTNPB (1×10⁻⁹ M) for RXR:RAR heterodimer assay or BRL 49633 (1×10⁻⁷ M) for the RXR:PPARγ heterodimer assay. Efficacy of the compounds of the present invention in the agonist and synergy assays was calculated as the maximum response obtained over the range of the dose response curve relative to the maximum response obtained by the reference agonist. Antagonist efficacy was determined as a function (%) of maximal inhibition.

RAR suppresses RXR ligand binding and transactivation of typical RXR agonists via allosteric interactions. Forman, B. M. et al., Cell, 81:541-550 (1995) and Kurokawa, R. et al., Nature 371:528-531 (1994). However, when RAR is occupied, typical RXR agonists activate the heterodimer. Forman, B. M. et al., Cell, 81:541-550 (1995) and Roy, B. et al., Mol. Cell. Biol., 15:6481-6487 (1995). To examine the effects of the compounds of the present invention on the transcriptional properties of the RXR:RAR heterodimer, a heterodimer cotransfection assay as described above was employed. Table 2 below shows the activity of selected compounds of the present invention in terms of agonist, antagonist or synergy efficacy in the RXR:RAR heterodimer cotransfection assay.

TABLE 2
Activity of RXR modulators of present invention in the
RXRα:RAR heterodimer cotransfection assays. Values
represent the mean of n > 2 independent experiments.

| | RXRα:RAR Heterodimer | | |
|---|---|---|---|
| | Cotransfection Assay | | |
| | Agonist | Antagonist | Synergy |
| | Efficacy | Efficacy | Efficacy |
| Example | (%)¹ | (%)² | (%)³ |
| 3 | 6 | — | 73 |
| 2 | 2 | 88 | 6 |
| 1 | 1 | 93 | 7 |
| 4 | 9 | 76 | 37 |
| 5 | 3 | 85 | 18 |

¹Efficacy calculated as maximal response relative to response of ATRA.
²Efficacy calculated relative to maximal repression (100%) in presence of 10 nM TTNPB.
³Efficacy calculated as maximal response in presence of 3 nM TTNPB relative to response to TTNPB alone.

ATRA and the RAR selective activator TTNPB strongly transactivate the RXR:RAR heterodimer. Compound 3 showed strong agonist activity in combination with TTNPB. Compounds 4 and 5 displayed weak to moderate agonist activity in combination with TTNPB. Compounds 1 and 2 were not active as RXR:RAR agonists alone or in combination with TTNPB, but rather displayed significant RXR:RAR antagonist activity as indicated by their efficacy in the antagonist assay.

In contrast to RAR, RXR:PPARγ heterodimers have previously been shown to be responsive to both RXR and PPAR ligands. Kliewer et al., Nature 358:771-774 (1992). To examine the effects of the compounds of the present invention on the transcriptional properties of the RXR:PPARγ, a heterodimer cotransfection assay as described above was employed. Table 3 below shows the activity of reference compounds and of selected compounds of the present invention in terms of agonist or synergy efficacy in the RXR:PPARγ heterodimer cotransfection assay. The compounds of the present invention, as was seen for the RXR:RAR heterodimer assay, also display a continuum of activities on the RXRx:PPARγ heterodimer. Compounds 2-5 display both agonist and synergistic activity. Compound 1 is a partial agonists alone and shows stronger activity in combination with the PPARγ ligand.

TABLE 3
Activity of RXR modulators of present invention in RXRα:PPARγ
heterodimer cotranfection assays. Values represent the mean of
n > 2 independent experiments.
RXRα:PPARγ
Heterodimer
Cotransfection
Assay

| | Agonist | Synergy |
|---|---|---|
| | Efficacy | Efficacy |
| Example | (%)¹ | (%)² |
| 3 | 42 | 182 |
| 2 | 32 | 150 |
| 1 | 11 | 25 |
| 4 | 53 | 233 |
| 5 | 55 | 107 |

¹Efficacy calculated as maximal response relative to response of the thiazolidinedione BRL49653.
²Efficacy calculated as maximal response in presence of 100 nM BRL49653 relative to response of BRL49653 alone.

Thus, although all of the compounds of the present invention directly and specifically bind RXR, they manifest distinct properties in the RXR:RXR homodimer assay as compared to the RXR:RAR and RXR:PPARγ heterodimer assays. The various RXR modulator compounds of the present invention have a range of activities when compared with each other and are truly dimer-selective RXR modulators, such that their actual function as either agonist, partial agonist and/or antagonist change depending upon the RXR partner and whether the partner is bound by ligand.

### Example 7

### Evaluation of Activity In Vivo

Rodents that are genetically defective in the leptin pathway are commonly used as animal models of non-insulin dependant diabetes mellitus (NIDDM). db/db mice and ZDF rats develop frank diabetes that progresses to include β-cell failure and the accompanying precipitous drop in plasma insulin levels. Both strains are profoundly obese, hyperglycemic, hyperinsulinemic, and hypertriglyceridemic. fa/fa rats, on the other hand, are obese and insulin resistant but do not develop frank diabetes and the associated hyperglycemia. All three rodent models were used to examine the efficacy of oral dosing with compounds of the invention on diabetes, insulin sensitivity, food consumption and body weight gain.

Mice (obtained from Jackson Laboratory), ZDF rats (obtained from Genetic Models Inc.) and fa/fa rats (obtained from either Charles River, or Harlan) are maintained on 12-hour light/dark cycle. Mice (age 28-42 days) are caged in groups of 5-6. Rats (age 7 weeks) are housed individually. All animals are allowed ad libitum access to water and food (Purina 5015 for mice and 5008 for rats). Compounds are administered at the specified doses by oral gavage on the morning of each day of any experiment. Blood samples are obtained 3 hours after dosing from fed animals under anesthesia and collected into heparinized capillary tubes from the tail vein.

Mice transgenic for the human apolipoprotein A-I gene (obtained from Jackson Laboratory) are used to evaluate PPARγ mediated effects on high density lipoprotein (HDL) cholesterol. The mice are handled as described above for db/db mice, except that they are fed Purina 5001.

Compounds that are full agonists at the RXR homodimer, such as LG100268, are efficacious insulin sensitizers in rodent models of NIDDM and, thus, lower blood glucose levels. However, such compounds raise triglycerides and suppress the thyroid hormone axis in these animals. On the other hand, full antagonists have no effect on glucose, triglycerides or the thyroid status in these same model systems. We have identified a specific subset of rexinoids that maintain the desirable insulin sensitizing activity and eliminate both the suppression of the thyroid axis and triglyceride elevations. These compounds are heterodimer selective modulators of RXR activity. They bind to RXR with high affinity (generally Kᵢ<50 nM) and produce potent synergistic activation of the RXR:PPARγ heterodimer. This synergistic activation of PPARγ in vitro is presumably a major determinant of the antidiabetic efficacy of compounds in vivo. To eliminate the undesirable increases in triglycerides and suppression of T4, the modulators must not significantly activate RXR:RAR heterodimers and must have substantial RXR:RAR antagonist activity.

When administered to obese, insulin resistant db/db mice (100 mg/kg by daily oral gavage for 14 days), compounds of the invention are expected to lower plasma glucose. However, unlike full agonists (e.g., LG100268), they are not expected to increase triglycerides.

Four week old db/db mice are essentially normoglycemic, they have not yet developed hyperglycemia. Treatment of such mice with a compound of the invention (30 mg/kg by daily oral gavage) is expected to prevent the development of hyperglycemia. This treatment is expected to successfully control plasma glucose levels for up to 11 weeks (when the mice are 15 weeks old).

Treatment of 7 week old db/db mice with metformin (300 mg/kg by daily oral gavage) lowers plasma glucose. However the maximum effect is seen following the first week of treatment. Over 3 subsequent weeks the efficacy of metformin decreases. At this point, treatment with metformin plus the addition of a compound of the invention (100 mg/kg by daily oral gavage) is expected to lowered plasma glucose to the level of age matched lean. Thus, the RXR modulator are expected to be efficacious in cases of secondary failure of metformin.

To determine whether compounds of the invention produce insulin sensitization, compounds of the invention can be administered to insulin resistant fa/fa rats (100 mg/kg by daily oral gavage for 14 days. In response to the oral glucose challenge, both insulin and glucose is expected to rise significantly less in animals treated with a compound of the invention than in untreated control animals. Animals treated with a compound of the invention are expected to consume the same amount of food and gain the same amount of weight as vehicle treated control animals. When fa/fa animals are treated with a thiazolinedione insulin sensitizer, they consume significantly more food and gain significantly more weight than control animals. In contrast, animals treated with a combination of the thiazolidinedione and a compound of the invention are expected to consume the same amount of food and gain the same amount of weight as the control animals. Compounds of the invention are expected to block the thiazolidinedione induced increases in both food consumption and body weight gain.

When administered to transgenic mice carrying the human apo A-I gene, compounds of the invention are expected to increase HDL cholesterol. However, unlike LG100268 which also raises triglycerides, compounds of the invention are not expected to raise triglycerides. Compounds of the invention that are not RXR:RAR heterodimer agonist and have greater than 50% RXR:RAR antagonists activity do not raise triglycerides in the transgenic mouse model, consistent with their heterodimer selectivity. This effect is consistent with activation of PPARα and, in fact, in vivo these compounds synergize with the weak PPARα agonist fenofibrate.

### Example 15

### Evaluation of Teratogenicity In Vivo

Teratogenicity is commonly evaluated by examination of fetuses obtained by cesarean section from pregnant mice dosed daily with test compound between gestation days 6-18. A blind study can be conducted using time-mated female Crl:CD-1® (ICR)BR mice to evaluate potential developmental toxicity (teratogenicity) following administration of a compound of the invention at either 30 or 200 mg/kg-day by daily oral gavage for the specified 12 days of gestation. Each test group consists of 7-8 pregnant females and produced approximately 100 live fetuses per test group. As a positive control, pregnant female mice are treated with the retinoid LG100268 at a dose of either 30 mg/kg-day or 100 mg/kg-day. Teratogenicity can be observed in fetuses from mice treated with the LG100268 at both dosage groups. In contrast, no teratogenic effects are expected to be observed in fetuses from mice treated with a compound of the invention. Compared to controls dosed with vehicle, no effects are expected to be observed on the number of Corpora lutea, implantation sites, live or dead fetuses, early or late resorptions, fetal weight or sex, gross external morphology or visceral morphology of the cranial region in fetuses from mice treated with a compound of the invention at either dose. The highest dose of a compound of the invention tested (200 mg/kg-day) is twice the dose required to produce maximum antidiabetic activity in db/db mice (100 mg/kg-day).

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. The compound represented by the following structural formula: 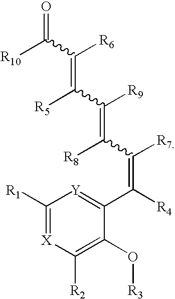
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy,
R₁₁ and R₁₂ are each, independently, H or an C₁-C₆ alkyl or taken together with the nitrogen to which they are attached form a heterocycle;
R₁₃ is H or a C₁-C₆ alky, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

2. The compound of claim 1, wherein X is N and Y is CH.

3. The compound of claim 1, wherein R₃ is an optionally substituted C₂-C₅ alkyl or a C₂-C₅ fluoroalkyl.

4. The compound of claim 1, wherein R₄ and R₇ are in a cis configuration.

5. The compound of claim 4, wherein R₅ and R₆ are in a trans configuration and R₈ and R₉ are in a trans configuration.

6. A compound selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

7. A pharmaceutical composition, comprising a pharmaceutically acceptable carrier and at least one compound represented by the following structural formula: 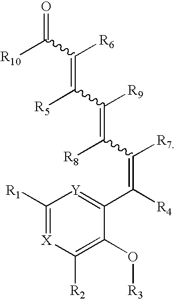
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alky, an aryl or an aralkyl; and
R₁₅ and R₁₆ are each, independently, H, a C₁-C₆ alkyl, an aryl or an aralkyl.

8. The pharmaceutical composition of claim 7, wherein X is N and Y is CH.

9. The pharmaceutical composition of claim 7, wherein R₃ is an optionally substituted C₂-C₅ alkyl or a C₂-C₅ fluoroalkyl.

10. The pharmaceutical composition of claim 7, wherein R₄ and R₇ are in a cis configuration.

11. The pharmaceutical composition of claim 10, wherein R₅ and R₆ are in a trans configuration and R₈ and R₉ are in a trans configuration.

12. A pharmaceutical composition, comprising a pharmaceutically acceptable carrier and at least one compound selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

13. A method for modulating retinoid X receptor activity in a mammal comprising administering to said mammal a pharmaceutically effective amount of at least one compound represented by the following structural formula: 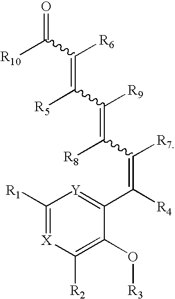
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

14. The method of claim 13, wherein X is N and Y is CH.

15. The method of claim 13, wherein R₃ is an optionally substituted C₂-C₅ alkyl or a C₂-C₅ fluoroalkyl.

16. The method of claim 13, wherein R₄ and R₇ are in a cis configuration.

17. The method of claim 16, wherein R₅ and R₆ are in a trans configuration and R₈ and R₉ are in a trans configuration.

18. The method of claim 13, wherein the compound is selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridinyl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

19. A method for modulating RXRα:PPARα heterodimer activity in a mammal comprising administering to said mammal a pharmaceutically effective amount of at least one compound represented by the following structural formula: 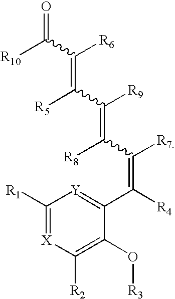
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₁₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each) independently, H, a C₁-C₆ alkyl, an aryl or an aralkyl.

20. The method of claim 19, wherein X is N and Y is CH.

21. The method of claim 19, wherein R₃ is an optionally substituted C₂-C₅ alkyl or a C₂-C₅ fluoroalkyl.

22. The method of claim 19, wherein R₄ and R₇ are in a cis configuration.

23. The method of claim 22, wherein R₅ and R₆ are in a trans configuration and R₈ and R₉ are in a trans configuration.

24. The method of claim 19, wherein the compound is selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

25. A method for modulating RXRa:PPARγ heterodimer activity in a mammal comprising administering to said mammal a pharmaceutically effective amount of at least one compound represented by the following structural formula: 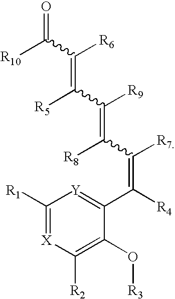
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy,
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

26. The method of claim 25, wherein X is N and Y is CH.

27. The method of claim 25, wherein R₃ is an optionally substituted C₂-C₅ alkyl or a C₂-C₅ fluoroalkyl.

28. The method of claim 25, wherein R₄ and R₇ are in a cis configuration.

29. The method of claim 28, wherein R₅ and R₆ are in a trans configuration and R₈ and R₉ are in a trans configuration.

30. The method of claim 25, wherein the compound is selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

31. A method for increasing HDL cholesterol levels and reducing triglyceride levels in a mammal comprising administering to said mammal a pharmaceutically effective amount of at least one compound represented by the following structural formula: 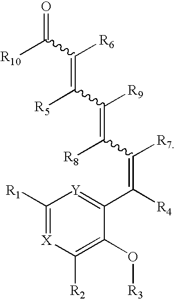
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

32. The method of claim 31, wherein X is N and Y is CH.

33. The method of claim 31, wherein R₃ is an optionally substituted C₂-C₅ alkyl or a C₂-C₅ fluoroalkyl.

34. The method of claim 31, wherein R₄ and R₇ are in a cis configuration.

35. The method of claim 34, wherein R₅ and R₆ are in a trans configuration and R₈ and R₉ are in a trans configuration.

36. The method of claim 31, wherein the compound is selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

37. A method for modulating lipid metabolizm in a mammal comprising administering to said mammal a pharmaceutically effective amount of at least one compound represented by the following structural formula: 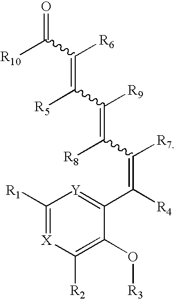
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

38. The method of claim 37, wherein X is N and Y is CH.

39. The method of claim 37, wherein R₃ is an optionally substituted C₂-C₅ alkyl or a C₂-C₅ fluoroalkyl.

40. The method of claim 37, wherein R₄ and R₇ are in a cis configuration.

41. The method of claim 40, wherein R₅ and R₆ are in a trans configuration and R₈ and R₉ are in a trans configuration.

42. The method of claim 37, wherein the compound is selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

43. A method for lowering blood glucose levels without altering serum triglyceride levels in a mammal comprising administering to said mammal a pharmaceutically effective amount of at least one compound represented by the following structural formula: 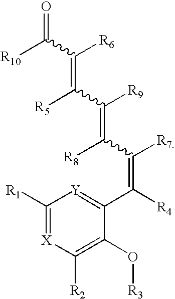
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

44. The method of claim 43, wherein X is N and Y is CH.

45. The method of claim 43, wherein R₃ is an optionally substituted C₂-C₅ alkyl or a C₂-C₅ fluoroalkyl.

46. The method of claim 43, wherein R₄ and R₇ are in a cis configuration.

47. The method of claim 46, wherein R₅ and R₆ are in a trans configuration and R₈ and R₉ are in a trans configuration.

48. The method of claim 43, wherein the compound is selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

49. A method treating or preventing a disease or condition selected from the group consisting of syndrome X, non-insulin dependent diabetes mellitus, cancer, photoaging, acne, psoriasis, obesity, cardiovascular disease, atherosclerosis, uterine leiomyomata, inflamatory disease, neurodegenerative diseases, wounds and baldness in a mammal comprising administering to said mammal a pharmaceutically effective amount of a compound represented by the following structural formula: 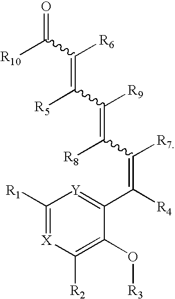
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

50. The method of claim 49, wherein X is N and Y is CH.

51. The method of claim 49, wherein R₃ is an optionally substituted C₂-C₅ alkyl or a C₂-C₅ fluoroalkyl.

52. The method of claim 49, wherein R₄ and R₇ are in a cis configuration.

53. The method of claim 52, wherein R₅ and R₆ are in a trans configuration and R₈ and R₉ are in a trans configuration.

54. The method of claim 49, wherein the compound is selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

55. A compound for use in therapy for a disorder modulated by a retinoid X receptor, a RXRα:PPARα heterodimer, or RXRa:PPARγ heterodimer, wherein the compound is represented by the following structural formula: 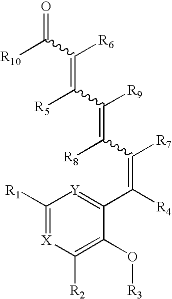
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

56. The method of claim 55, wherein X is N and Y is CH.

57. The method of claim 55, wherein R₃ is an optionally substituted C₂-C₅ alkyl or a C₂-C₅ fluoroalkyl.

58. The method of claim 55, wherein R₄ and R₇ are in a cis configuration.

59. The method of claim 58, wherein R₅ and R₆ are in a trans configuration and R₈ and R₉ are in a trans configuration.

60. The method of claim 55, wherein the compound is selected from the group consisting of:
7-(3-butoxy-2,6-diisopropyl-pyridin-4-yl)-3-methyl-octa-2(E),4(E),6(Z)-trienoic acid;
7-(2,6-diisopropyl-3-propoxy-pyridin-4-yl)-3-methyl-octa-2(E),4(E),6(Z)-trienoic acid;
7-(2,6-diisopropyl-3-ethoxy-pyridin-4-yl)-3-methyl-octa-2(E),4(E),6(Z)-trienoic acid;
7-[3-(2,2-difluoro-ethoxy)-2,6-diisopropyl-pyridin-4-yl]-3-methyl-octa-2(E),4(E),6(Z)-trienoic acid; and
7-[2,6-diisopropyl-3-(2,2,2-trifluoro-ethoxy)-pyridin-4-yl]-3-methyl-octa-2(E),4(E),6(Z)-trienoic acid,
and pharmaceutically acceptable salts, solvates and hydrates thereof.

61. Use of a compound for the manufacture of a medicament for the treatment of a condition modulated by a retinoid X receptor, a
RXRα:PPARα heterodimer, or RXRα:PPARγ heterodimer, wherein the compound is represented by the following structural formula: structural formula: 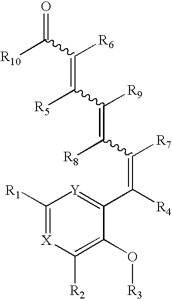
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R₁₀ is OR₁₃, OC(O)R₁₄, NR₁₅R₁₆ or an aminoalkoxy;
R₁₁ and R₁₂ are each, independently, H or an C₁-C₆ alkyl or taken together with the nitrogen to which they are attached form a heterocycle;
R₁₃ is H or a C₁-C₆ alkyl, an aryl or an aralkyl;
R₁₄ is a C₁-C₆ alkyl, an aryl or an aralkyl; and
R₁₅ and R₁₆ are each, independently, H, a C₁-C₆ alkyl, an aryl or an aralkyl.

62. A method of preparing a 7-(substituted azaaryl)-hepta-2,4,6-trienoic acid alkyl ester represented by the following structural formula: 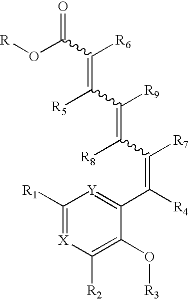
and geometrical isomers and pharmaceutically acceptable salts, solvates and hydrates thereof, wherein:
X and Y are each, independently, CH or N, wherein at least one of X or Y is N;
R₁ and R₂ are each, independently, H, an optionally substituted C₁-C₆ alkyl, C₁-C₆ haloalkyl, an optionally substituted heteroalkyl, an optionally substituted C₃-C₇ cycloalkyl, an optionally substituted C₂-C₆ alkenyl, C₂-C₆ haloalkenyl, a heteroalkenyl, an optionally substituted C₂-C₆ alkynyl, C₂-C₆ haloalkynyl, an aryl, a heteroaryl, a C₁-C₆ alkoxy, an aryloxy, or an amino group represented by the formula NR₁₁R₁₂;
R₃ is an optionally substituted C₁-C₉ alkyl, a C₁-C₆ haloalkyl, an optionally substituted C₃-C₇ cycloalkyl, or an optionally substituted aralkyl;
R₄ and R₅ are each, independently, H, F, an optionally substituted C₁-C₃ alkyl, or a C₁-C₃ haloalkyl;
R₆, R₇, R₈, and R₉ are each, independently, H or F;
R is a C₁-C₆ alkyl; and
R₁₁ and R₁₂ are each, independently, H or an C₁-C₆ alkyl or taken together with the nitrogen to which they are attached form a heterocycle, wherein R₄ and R₇ are in a cis configuration, comprising the steps of:
a) heating an acyl-hydroxyazaaryl represented by the following structural formula: 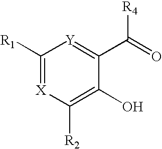
with a (carbalkoxymethylene) triphenylphosphorane represented by the following structural formula: 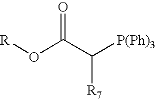
to form a substituted azacoumarin represented by the following structural formula: 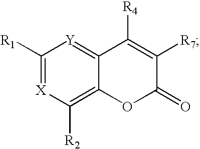
b) treating the azacoumarin with a reducing agent to form a 3-(hydroxy-azaaryl)-prop-2-en-1-ol represented by the following structural formula: 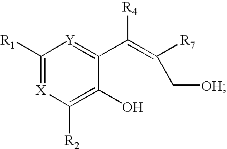
a. reacting the 3-(hydroxy-azaaryl)-prop-2-en-1-ol with an aliphatic halide represented by the formula R₃—X in the presence of cesium fluoride or cesium carbonate to form an optionally substituted 3-(alkoxy-azaaryl)-prop-2-en-1-ol represented by the following structural formula: 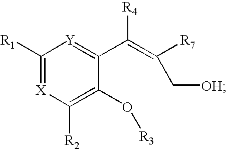
b. oxidizing the 3-(alkoxy-azaaryl)-prop-2-en-1-ol with Dess-Martin periodinane to form a 3-(alkoxy-azaaryl)-prop-2-en-1-al represented by the following structural formula: 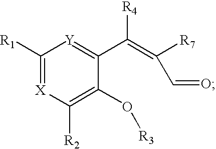
c. treating a trialkyl phosphocrotonate represented by the following structural formula: 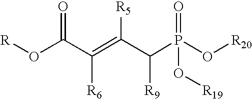
with an alkyl lithium to form an anion;
d. reacting the anion of the trialkyl phosphocrotonate with the 3-(alkoxy-azaaryl)-prop-2-en-1-al to form said 7-(substituted azaaryl)-hepta-2,4,6-trienoic acid alkyl ester.

63. The method of claim 62, further comprising the step of treating the 7-(substituted azaaryl)-hepta-2,4,6-trienoic acid alkyl ester with an alkali metal hydroxide to form a 7-(substituted azaaryl)-hepta-2,4,6-trienoic acid.
